(19) United States
(12) Patent Application Publication
(10) Pub. No.: US 2014/0127290 A1
(43) Pub. Date: May 8, 2014

(54) Microcapsules Encapsulating Living Cells

(71) Applicant: OHIO STATE INNOVATION FOUNDATION, Columbus, OH (US)

(72) Inventors: Xiaoming He, Dublin, OH (US); Pranay Agarwal, Columbus, OH (US); Shuting Zhao, Columbus, OH (US)

(73) Assignee: OHIO STATE INNOVATION FOUNDATION, Columbus, OH (US)

(21) Appl. No.: 14/075,912

(22) Filed: Nov. 8, 2013

Related U.S. Application Data
(60) Provisional application No. 61/724,126, filed on Nov. 8, 2012.

(51) Int. Cl. C12N 11/10; A61K 35/12

(52) U.S. Cl. CPC C12N 11/10; A61K 35/12
USPC 424/451; 424/93.7; 435/283.1; 435/178

(57) ABSTRACT

Disclosed are devices and methods for encapsulating living cells, microencapsulated cells produced by the disclosed devices and methods, as well as methods of using the disclosed microencapsulated cells.

### CROSS REFERENCE TO RELATED APPLICATIONS

This application claims the benefit of U.S. Provisional Application No. 61/724,126, filed Nov. 8, 2012, which is hereby incorporated herein by reference in its entirety.

### STATEMENT REGARDING FEDERALLY SPONSORED RESEARCH OR DEVELOPMENT

This invention was made with Government Support under Grant No. R01 EB012108 awarded by the National Institutes of Health, and Grant No. CBET-1154965 awarded by the National Science Foundation. The Government has certain rights in the invention.

### TECHNICAL FIELD

This application relates generally to devices and methods for cell microencapsulation.

### BACKGROUND

Both pluripotent and multipotent stem cells have been proposed as promising cell-based medicine for treating various tissue and organ dysfunctions as they can be directed to differentiate into various specific lineages to facilitate tissue regeneration (Langer R, Vacanti J P. Science 1993 260:920-926; Gearhart J. Science 1998; 282: 1061-1062; Stappenbeck T S, Miyoshi H. Science 2009; 324(5935): 1666-9). With much attention being focused on identifying and optimizing chemical cues for differentiation in the past years however, the practice of stem cell culture and differentiation to date has been done primarily either in hanging drops (for pluripotent stem cells only) or on the 2D substrate of Petri dish, which is very different from the native 3D milieu of stem cells in the body.

Stem cells encapsulated in microcapsules of natural polymers such as alginate and collagen have been proposed as a promising cell-based medicine for arthritis, cardiac infarction, and Parkinson's and Alzheimer diseases (Zimmermann, H., et al. Curr Diab Rep 2007 7(4):314-320). Encapsulated stem cells can be induced to differentiate into desired cell types such as chondrocytes, cardiac cells, and neurons can promote rapid regeneration of normal tissue to replace the diseased one. However, differentiation of stem cells to a specific cell type is not straightforward because factors such as cell-cell and cell-substrate interactions play an important role in their proliferation and differentiation. These interactions, however, with recent progress in microtissue engineering, can be fine-tuned by regulating the stiffness and size of the microcapsules or density of the encapsulated cells (Watt F M., et al. Science 2000 287(5457):1427-1430; Park, J., et al. Lab Chip. 2007 August; 7(8):1018-28).

Encapsulation of cells and tissues from the reproductive system including oocytes and embryos in a hydrogel matrix such as that of alginate is gaining more and more attention for improving the outcome of assisted reproduction (West E R, et al. Biomaterials 2007 28(30):4439-48; Torre, M L, et al. Recent Pat Drug Deliv Formul 2007 1(1):81-5). Moreover, a recent study indicates that encapsulating mesenchymal stem cells (MSCs) in alginate hydrogel microcapsules could allow effective cryopreservation of the cells by vitrification using a low and non-toxic concentration of cryoprotectants (Zhang, W, et al. Biomed Microdevices 2010; 12(1):89-96). This idea of augmenting cell vitrification by microencapsulation however, has not been tested for mammalian oocytes. Moreover, encapsulation of mammalian oocytes in the tiny hydrogel microcapsules is much more challenging because of their big size (˜130 μm for human oocytes compared to ˜10 μm for MSCs) and high sensitivity to stress.

### SUMMARY

Disclosed are devices and methods that allow the 3D culture of living cells in a submillimeter space that ensure sufficient supply of oxygen and nutrients for the survival for all cells. Encapsulation of living cells can be achieved in microcapsules in one step of a mild process to ensure high immediate cell viability. Moreover, the microenvironment of the cells in the microcapsules can be controlled, which is of utmost importance for regulating proliferation, migration, differentiation, and gene and protein expression of living cells (particularly stem cells) for tissue regeneration and cell-based therapy. The microcapsules can also be used to encapsulate biomolecules to achieve the desired sustained release for therapeutic applications.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic of a microfluidic device.

FIG. 2 is a schematic of a microfluidic device.

FIG. 3 is a schematic of a microfluidic device.

FIG. 4 is a schematic of a microfluidic device

FIGS. 5A-5C are time lapse images showing the process of microcapsule formation in a microfluidic device. FIGS. 5D and 5E are phase contrast images (10×) showing microcapsules of various sizes could be obtained by varying the flow rates of core, shell, and oil solutions. In FIG. 5D, 250 μm microcapsules were obtained using core, shell, and oil solutions flow rates or 90, 200 and 4000 μl/hr, respectively. In FIG. 5E, 175 um microcapsules were obtained using core, shell, and oil solutions flow rates or 90, 200 and 8000 μl/hr, respectively. FIG. 5F is a histogram depicting size distribution of microcapsules with oil flow rates of 4 ml/hr (242 μm mean, SD 23 μm) or 8 ml/hr (181.1 μm mean, SD: 10.6 μm).

FIGS. 6A-6C are confocal images of bottom, middle and top planes of microcapsule stained with FITC IgG, showing the core-shell structure of the microcapsule. FIG. 6D is a phase contrast image of collagen core microcapsules stained with FITC. FIGS. 6E-6F are phase contrast (FIG. 6E) and fluorescent (FIG. 6F) images of the collagen gel in the microcapsule core after alginate shell of the microcapsule is removed using sodium citrate.

FIGS. 7A and 7B are phase contrast images of microcapsules obtained at low core fluid flow rate (FIG. 7A) or high core fluid flow rate (FIG. 7B).

FIGS. 8A-8B are phase contrast images of R1 ES cells encapsulated in core-shell microcapsules on day 0. FIG. 8C is a fluorescent image of these cells stained for living cells showing high viability (>92%) of the encapsulated cells. FIGS. 8D-8E are phase contrast images of the encapsulated cells on day 3. FIG. 8F is a histogram depicting number of cells encapsulated per microcapsule. FIGS. 8G-8H are phase contrast images showing formation of single EBs at day 9.

FIGS. 9A-9B are phase contrast (FIG. 9A) and fluorescent OCT4 staining (FIG. 9B) of microencapsulated ES cell aggregates at day 9 to verify pluripotency. FIG. 9C is a graph showing expression of pluripotent genes OCT4, SOX2, NANOG, and KLF2 between control ES cells (left bars) and ES cell aggregates (right bars) formed in core-shell microcapsules by real time reverse transcription (RT) PCR.

FIGS. 10A-10C are time lapse images of a porcine oocyte (black dot) undergoing microencapsulation in the microfluidic channel. FIGS. 10D-10E are phase contrast images of non-encapsulated porcine oocytes (FIG. 10D) and oocytes encapsulated in alginate core-shell microcapsules (FIG. 10E). FIG. 10F is a fluorescent image of encapsulated oocytes stained for living cells.

FIG. 11 is a schematic of coaxial electrospray setup for producing core-shell microencapsulated cells.

FIGS. 12A-12B are images of microcapsules with 100 μm core and 200 μm overall size fabricated in high voltage field (FIG. 12A) and 300 μm core and 400 μm overall size fabricated in low voltage field (FIG. 12B). FIGS. 12C-12D are images of R1 mouse embryonic stem cells encapsulated in large microcapsules as single cells (FIG. 12C) that proliferated to form a single cell aggregate in each microcapsule after 7 days (FIG. 12D).

FIGS. 13A-13N illustrate the survival and proliferation of embryonic stem (ES) cells encapsulated in microcapsules with a hydrogel (FIGS. 13A-13F) and aqueous liquid (FIGS. 13G-13N) core. Phase contrast and fluorescence micrographs illustrate the high viability of all encapsulated ES cells on day 0 (FIGS. 13A, 13D, 13G, and 13J), the higher viability of ES cells in the liquid than hydrogel core on days 3 (FIGS. 13B, 13E, 13H and 13K) and 7 (FIGS. 13C, 13F, 13I, 13L, 13M and 13N), and the formation of either multiple ES cell aggregates of non-uniform size and irregular shape in hydrogel core (FIGS. 13C and 13F) or single spheroidal aggregates of uniform size with high viability in liquid core (FIGS. 13I, 13L and 13M-13N) on day 7.

FIGS. 14A-D show pluripotency of ES cell aggregates/colonies. FIG. 14A is qRT-PCR data of pluripotency genes showing that the aggregated ES cells in 3D liquid core have significantly higher expression of Klf2 and Nanog compared to the cells under 2D culture and significantly higher expression of klf2, Nanog, and Sox-2 than that in 3D hydrogel core. FIG. 14B is immunoblotting data of pluripotency protein markers together with GAPDH, the house-keeping protein marker. FIG. 14C is quantitative analyses of the data in FIG. 14B indicate aggregates formed in the 3D liquid core have significantly higher expression of Nanog and Sox-2 while the expression of Oct-4 is not significantly different under the three different culture conditions. FIG. 14D is immunohistochemical staining of Oct-4, SSEA-1, and cell nuclei together with differential interference contrast (DIC) micrographs showing high expression of the pluripotency marker proteins in the aggregated ES cells formed in the 3D liquid core. *: p<0.05 and **: p<0.01.

FIGS. 15A-15D show cardiac differentiation of the ES cell aggregates/colonies induced by BMP-4 and bFGF. FIG. 15A is a graph showing cumulative percentage of beating foci on each day after differentiation, revealing that aggregates from the 3D liquid core has significantly more beating than that from 3D hydrogel and 2D culture. FIG. 15B is flow cytometry data showing approximately 17%, 33.4%, and 42.2% of cells differentiated from aggregates or colonies obtained under 2D, 3D hydrogel, and 3D liquid culture are positive for cTnT, a protein marker specific of cardiomyocytes. FIG. 15C is qRT-PCR data showing that cardiac specific genes including Nkx2.5 and cTnT (but not the brachyury or T gene for early mesoderm) are significantly up-regulated in cells differentiated from ES cell aggregates obtained in the 3D liquid core compared to all the other culture conditions while cells differentiated from ES cell aggregates obtained in the 3D hydrogel core have significantly highest expression of the early mesoderm marker protein Brachyury (or T). FIG. 15D is a series of confocal fluorescence micrographs of immunohistochemical staining of the cTnT (top) together with two other proteins important for the proper function of cardiomyocytes: α-actinin (middle) in cardiac sarcomere and connexin 43 (Cx43, bottom) in gap junction. *: p<0.05 and **: p<0.01.

FIGS. 16A-16B show that cancer stem cells (CSCs) enriched by microencapsulation using the aqueous liquid core and alginate hydrogel shell are much more tumorigenic, compared to that obtained using the conventional approach using expensive ultralow attachment plate. FIGS. 16A and 16B show tumor incidence (%) (FIG. 16A, P value=0.0016, n=4) and tumor volume (mm³) (FIG. 16B, P value=0.0053, n=4) in mice injected with 3,000 cells from the following conditions: PC-3 control CSCs, CSCs after convention culture for 10 days or 2 days, and encapsulated CSCs cultured for 2 days.

FIG. 17 is qRT-PCR data of pluripotency genes showing that the aggregated CSCs cells in 3D liquid core have significantly higher expression of pluripotency genes compared to PC-3 cells or CSCs cultured using conventional methods.

### DETAILED DESCRIPTION

Disclosed are devices and methods for encapsulating living cells, microencapsulated cells produced by this method, as well as methods of using these microencapsulated cells for 3D cell culture, differentiation, and tissue engineering.

### DEFINITIONS

“Microcapsule” refers to a particle or capsule having a mean diameter of about 50 μm to about 1000 μm, formed of a cross-linked hydrogel shell surrounding a biocompatible matrix. The microcapsule may have any shape suitable for cell encapsulation. The microcapsule may contain one or more cells dispersed in the biocompatible matrix, cross-linked hydrogel, or combination thereof, thereby “encapsulating” the cells.

“Hydrogel” refers to a substance formed when an organic polymer (natural or synthetic) is cross-linked via covalent, ionic, or hydrogen bonds to create a three-dimensional open-lattice structure which entraps water molecules to form a gel. Biocompatible hydrogel refers to a polymer that forms a gel which is not toxic to living cells, and allows sufficient diffusion of oxygen and nutrients to the entrapped cells to maintain viability.

“Alginate” is a collective term used to refer to linear polysaccharides formed from β-D-mannuronate and β-L-guluronate in any M/G ratio, as well as salts and derivatives thereof.

“Biocompatible” generally refers to a material and any metabolites or degradation products thereof that are generally non-toxic to the recipient and do not cause any significant adverse effects to the subject.

“Cell” refers to any living cell. The cell may be xenogeneic, autologous, or allogeneic. The cell can be a primary cell obtained directly from a plant or animal, such as a mammal. The cell may also be a cell derived from the culture and expansion of a cell obtained from a plant or animal. For example, the cell may be a stem cell. Immortalized cells are also included within this definition. In some embodiments, the cell has been genetically engineered to express a recombinant protein and/or nucleic acid.

“Microfluidic Device” refers to a device that includes one or more microfluidic channels, one or more microfluidic valves, one or more microfluidic chambers, or combinations thereof, and are configured to carry, store, transport, combine, and/or react component solutions in fluid volumes of less than ten milliliters (e.g., in fluid volumes of 5 mL or less, in fluid volumes of 2.5 mL or less, or in fluid volumes of 1.0 mL or less) to form microcapsules.

“Microfluidic Channel” refers to a feature within a microfluidic device that forms a path, such as a conduit, through which one or more fluids can flow. In some embodiments, microfluidic channels have at least one cross-sectional dimension that is in the range from about 0.1 microns to about 750 microns (e.g., from about 1 micron to about 750 microns, from about 1 micron to about 500 microns, from about 10 microns to about 500 microns, or from about 50 microns to about 450 microns).

Microcapsules

Disclosed are microcapsules containing a core and a shell (core-shell microcapsules) produced in a single step via a mild process that ensures high immediate cell viability. The core can contain mammalian cell or cell aggregate suspended or encapsulated in a matrix. For example, the matrix can be a viscous aqueous liquid or a hydrogel. In some embodiments, the matrix contains proteins suitable for promoting a cell activity, such as survival, attachment, growth, pluripotency, or differentiation. For example, the protein can be collagen, fibrin, gelatin, elastin, or elastin-like polypeptides (ELPs), or a derivative thereof.

The shell surrounding the core can be a biocompatible hydrogel. One advantage of the disclosed microcapsules is the ability of the matrix and the biocompatible hydrogel to be distinct in their chemical composition or concentration. For example, the matrix can be an aqueous liquid while the shell can be a hydrogel surrounding the liquid core.

In some embodiments, the core and the shell can be formed so as to have distinct physical properties. For example, the core and the shell of the microcapsules can be fabricated to each have a different modulus of elasticity, density, polarity, thickness, hydrophobicity/hydrophilicity, or combinations thereof.

Examples of materials which can be used to form a suitable hydrogel (e.g. in the core, shell, or combinations thereof) include polysaccharides such as alginate, polyphosphazines, poly(acrylic acids), poly(methacrylic acids), poly(alkylene oxides), poly(vinyl acetate), poly(acrylamides) such as poly(N-isopropylacrylamide), polyvinylpyrrolidone (PVP), and copolymers and blends of each. See, for example, U.S. Pat. Nos. 5,709,854, 6,129,761 and 6,858,229. In some embodiments, block copolymers can be used. For example, poloxamers containing a hydrophobic poly(alkylene oxide) segment (i.e., polypropylene oxide) and hydrophilic poly(alkylene oxide) segment (i.e., polyethylene oxide) can be used. Polymers of this type are available are known in the art, and commercially available under the trade name PLURONICS from BASF. In some embodiments, the material is selected such that it forms a thermally responsive hydrogel.

In general, the polymers used to form the core and shell are at least partially soluble in aqueous solutions, such as water, buffered salt solutions, or aqueous alcohol solutions. In some embodiments, the polymers have polar groups, charged groups, acidic groups or salts thereof, basic groups or salts thereof, or combinations thereof. Examples of polymers with acidic groups poly(phosphazenes), poly(acrylic acids), poly(methacrylic acids), poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups include carboxylic acid groups and sulfonic acid groups.

Examples of polymers with basic groups include poly(vinyl amines), poly(vinyl pyridine), poly(vinyl imidazole), and some imino substituted polyphosphazenes. Nitrogen-containing groups in these polymers can be converted to ammonium or quaternary salts. Ammonium or quaternary salts can also be formed from the backbone nitrogens or pendant imino groups. Examples of basic groups include amino and imino groups.

In certain embodiments, the biocompatible hydrogel-forming polymer is a water-soluble gelling agent. In certain embodiments, the water-soluble gelling agent is a polysaccharide gum, such as a polyanionic polysaccharide. In some cases, cells or cell aggregates are encapsulated using an anionic polymer such as alginate to form a microcapsule shell, core, or combinations thereof.

In some embodiments, the matrix comprises a viscous aqueous solution. For example, in some embodiments, the matrix can have a viscosity that is at least two times, four times, six times, eight times, ten times, or twenty times the viscosity of water at 25° C. In certain embodiments, the matrix can have a viscosity that is at least two times, four times, six times, eight times, ten times, or twenty times the viscosity of ethylene glycol at 25° C.

In some cases, cells or cell aggregates are encapsulated using an anionic polymer such as alginate to form a hydrogel matrix (e.g., core). The hydrogel matrix can optionally be crosslinked, if desired. The matrix (e.g., the core) can also be formed from viscous solutions, such as, for example solutions of cellulose and its derivatives (e.g., carboxymethyl cellulose).

Mammalian and non-mammalian polysaccharides have been explored for cell encapsulation. These materials can be used, alone or in part, to form the core, the shell, or both the core and the shell. Exemplary polysaccharides include alginate, chitosan, hyaluronan (HA), and chondroitin sulfate. Alginate and chitosan form crosslinked hydrogels under certain solution conditions, while HA and chondroitin sulfate are preferably modified to contain crosslinkable groups to form a hydrogel.

In some embodiments, the core, the shell, or both the core and the shell comprise alginate or derivative thereof. Alginates are a family of unbranched anionic polysaccharides derived primarily from brown algae which occur extracellularly and intracellularly at approximately 20% to 40% of the dry weight. The 1,4-linked α-1-guluronate (G) and β-D-mannuronate (M) are arranged in homopolymeric (GGG blocks and MMM blocks) or heteropolymeric block structures (MGM blocks). Cell walls of brown algae also contain 5% to 20% of fucoidan, a branched polysaccharide sulphate ester with 1-fucose four-sulfate blocks as the major component. Commercial alginates are often extracted from algae washed ashore, and their properties depend on the harvesting and extraction processes. Although the properties of the hydrogel can be controlled to some degree through changes in the alginate precursor (molecular weight, composition, and macromer concentration), alginate does not degrade, but rather dissolves when the divalent cations are replaced by monovalent ions. In addition, alginate does not promote cell interactions.

Alginate can form a gel in the presence of divalent cations via ionic crosslinking Crosslinking can be performed by addition of a divalent metal cation (e.g., a calcium ion or a barium ion), or by cross-linking with a polycationic polymer (e.g., an amino acid polymer such as polylysine). See e.g., U.S. Pat. Nos. 4,806,355, 4,689,293 and 4,673,566 to Goosen et al.; U.S. Pat. Nos. 4,409,331, 4,407,957, 4,391,909 and 4,352,883 to Lim et al.; U.S. Pat. Nos. 4,749,620 and 4,744,933 to Rha et al.; and U.S. Pat. No. 5,427,935 to Wang et al. Amino acid polymers that may be used to crosslink hydrogel forming polymers such as alginate include the cationic poly(amino acids) such as polylysine, polyarginine, polyornithine, and copolymers and blends thereof.

In certain embodiments, the core, the shell, or both the core and the shell comprise alginate or derivative thereof in combination with a protein (e.g., collagen or derivatives thereof or fibrin or derivatives thereof). In certain embodiments, the biocompatible, hydrogel-forming polymer used to form the shell is alginate or derivative thereof.

In some embodiments, the core, the shell, or both the core and the shell comprise chitosan or derivative thereof. Chitosan is made by partially deacetylating chitin, a natural non-mammalian polysaccharide, which exhibits a close resemblance to mammalian polysaccharides, making it attractive for cell encapsulation. Chitosan degrades predominantly by lysozyme through hydrolysis of the acetylated residues. Higher degrees of deacetylation lead to slower degradation times, but better cell adhesion due to increased hydrophobicity. Under dilute acid conditions (pH<6), chitosan is positively charged and water soluble, while at physiological pH, chitosan is neutral and hydrophobic, leading to the formation of a solid physically crosslinked hydrogel. The addition of polyol salts enables encapsulation of cells at neutral pH, where gelation becomes temperature dependent.

Chitosan has many amine and hydroxyl groups that can be modified. For example, chitosan has been modified by grafting methacrylic acid to create a crosslinkable macromer while also grafting lactic acid to enhance its water solubility at physiological pH. This crosslinked chitosan hydrogel degrades in the presence of lysozyme and chondrocytes. Photopolymerizable chitosan macromer can be synthesized by modifying chitosan with photoreactive azidobenzoic acid groups. Upon exposure to UV in the absence of any initiator, reactive nitrene groups are formed that react with each other or other amine groups on the chitosan to form an azo crosslink.

In some embodiments, the core, the shell, or both the core and the shell comprise hyaluronan or derivative thereof. Hyaluronan (HA) is a glycosaminoglycan present in many tissues throughout the body that plays an important role in embryonic development, wound healing, and angiogenesis. In addition, HA interacts with cells through cell-surface receptors to influence intracellular signaling pathways. Together, these qualities make HA attractive for tissue engineering scaffolds. HA can be modified with crosslinkable moieties, such as methacrylates and thiols, for cell encapsulation. Crosslinked HA gels remain susceptible to degradation by hyaluronidase, which breaks HA into oligosaccharide fragments of varying molecular weights. Auricular chondrocytes can be encapsulated in photopolymerized HA hydrogels where the gel structure is controlled by the macromer concentration and macromer molecular weight. In addition, photopolymerized HA and dextran hydrogels maintain long-term culture of undifferentiated human embryonic stem cells. HA hydrogels have also been fabricated through Michael-type addition reaction mechanisms where either acrylated HA is reacted with PEG-tetrathiol, or thiol-modified HA is reacted with PEG diacrylate.

Chondroitin sulfate makes up a large percentage of structural proteoglycans found in many tissues, including skin, cartilage, tendons, and heart valves, making it an attractive biopolymer for a range of tissue engineering applications. Photocrosslinked chondroitin sulfate hydrogels can be been prepared by modifying chondroitin sulfate with methacrylate groups. The hydrogel properties were readily controlled by the degree of methacrylate substitution and macromer concentration in solution prior to polymerization. Further, the negatively charged polymer creates increased swelling pressures allowing the gel to imbibe more water without sacrificing its mechanical properties. Copolymer hydro gels of chondroitin sulfate and an inert polymer, such as PEG or PVA, may also be used.

In some embodiments, the core, the shell, or both the core and the shell comprise a synthetic polymer or polymers. Polyethylene glycol (PEG) has been the most widely used synthetic polymer to create macromers for cell encapsulation. A number of studies have used poly(ethylene glycol)di(meth)acrylate to encapsulate a variety of cells. Biodegradable PEG hydrogels can be been prepared from triblock copolymers of poly(α-hydroxy esters)-b-poly(ethylene glycol)-b-poly(α-hydroxy esters) endcapped with (meth)acrylate functional groups to enable crosslinking PLA and poly(8-caprolactone) (PCL) have been the most commonly used poly(α-hydroxy esters) in creating biodegradable PEG macromers for cell encapsulation. The degradation profile and rate are controlled through the length of the degradable block and the chemistry. The ester bonds may also degrade by esterases present in serum, which accelerates degradation. Biodegradable PEG hydrogels can also be fabricated from precursors of PEG-bis-[2-acryloyloxy propanoate]. As an alternative to linear PEG macromers, PEG-based dendrimers of poly(glycerol-succinic acid)-PEG, which contain multiple reactive vinyl groups per PEG molecule, can be used. An attractive feature of these materials is the ability to control the degree of branching, which consequently affects the overall structural properties of the hydrogel and its degradation. Degradation will occur through the ester linkages present in the dendrimer backbone.

The biocompatible, hydrogel-forming polymer can contain polyphosphoesters or polyphosphates where the phosphoester linkage is susceptible to hydrolytic degradation resulting in the release of phosphate. For example, a phosphoester can be incorporated into the backbone of a crosslinkable PEG macromer, poly(ethylene glycol)-di-[ethylphosphatidyl (ethylene glycol) methacrylate] (PhosPEG-dMA), to form a biodegradable hydrogel. The addition of alkaline phosphatase, an ECM component synthesized by bone cells, enhances degradation. The degradation product, phosphoric acid, reacts with calcium ions in the medium to produce insoluble calcium phosphate inducing autocalcification within the hydrogel. Poly(6-aminoethyl propylene phosphate), a polyphosphoester, can be modified with methacrylates to create multivinyl macromers where the degradation rate was controlled by the degree of derivitization of the polyphosphoester polymer.

Polyphosphazenes are polymers with backbones consisting of nitrogen and phosphorous separated by alternating single and double bonds. Each phosphorous atom is covalently bonded to two side chains. The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Hydrolytically stable polyphosphazenes are formed of monomers having carboxylic acid side groups that are crosslinked by divalent or trivalent cations such as Ca²⁺ or Al³⁺. Polymers can be synthesized that degrade by hydrolysis by incorporating monomers having imidazole, amino acid ester, or glycerol side groups. Bioerodible polyphosphazines have at least two differing types of side chains, acidic side groups capable of forming salt bridges with multivalent cations, and side groups that hydrolyze under in vivo conditions, e.g., imidazole groups, amino acid esters, glycerol and glucosyl. Hydrolysis of the side chain results in erosion of the polymer. Examples of hydrolyzing side chains are unsubstituted and substituted imidizoles and amino acid esters in which the group is bonded to the phosphorous atom through an amino linkage (polyphosphazene polymers in which both R groups are attached in this manner are known as polyaminophosphazenes). For polyimidazolephosphazenes, some of the “R” groups on the polyphosphazene backbone are imidazole rings, attached to phosphorous in the backbone through a ring nitrogen atom.

Another advantage of the disclosed methods of encapsulating cells is the ability to simultaneously encapsulate cells within both the shell and the core. In particular, the cells in the shell can be distinct from the cells in the core. This is particularly useful for 3D culture and tissue engineering. For example, in some embodiments, the core could contain endothelial cells, and the shell could contain smooth muscle cells.

The core and shell can therefore also contain proteins, such as proteins suitable for promoting cell survival, attachment, growth, pluripotency, or differentiation. Therefore, in some embodiments, the core, shell, or combination thereof further comprises collagen, fibrin, gelatin, elastin, or elastin-like polypeptides (ELPs), or a derivative thereof. In some embodiments, only the shell further comprises collagen, fibrin, gelatin, elastin, or elastin-like polypeptides (ELPs), or a derivative thereof. In some embodiments, only the core further comprises collagen, fibrin, gelatin, elastin, or elastin-like polypeptides (ELPs), or a derivative thereof.

The cells or cell aggregates encapsulated in the core and/or shell of the disclosed microcapsules can be any living cell type, including, but not limited to, a keratinizing epithelial cells, wet stratified barrier epithelial cells, exocrine secretory epithelial cells, hormone secreting cells, epithelial absorptive cells (gut, exocrine glands and urogenital tract), metabolism and storage cells, barrier function cells (lung, gut, exocrine glands and urogenital tract), epithelial cells lining closed internal body cavities, ciliated cells with propulsive function, extracellular matrix secretion cells, contractile cells, blood and immune system cells, sensory transducer cells, autonomic neuron cells, sense organ and peripheral neuron supporting cells, central nervous system neurons and glial cells, lens cells, pigment cells, germ cells, and nurse cells. Also included are any stem cells and progenitor cells of the cells disclosed herein, as well as the cells they lead to. The cells can be pluripotent stem cells, multipotent stem cells, progenitor cells, primary cells, or gametes. The cells can be a mixture of single cells or cell aggregates, such as antral or pre-antral follicles, or native tissue from other organs.

In certain embodiments, the cells are pluripotent cells, including both embryonic stem cells and induced pluripotent stem cells (iPSC). In certain embodiments, the cells are mesenchymal stem cells, hematopoietic stem cells, fibroblasts, endothelial cells, pericytes, astrocytes, macrophages, or combinations thereof.

In certain embodiments, the cells are cancer stem cells (CSCs), such as CSCs isolated from a tumor. In these embodiments, the disclosed method can be used to enrich the CSCs, which would be advantageous for identifying effective therapies both in research lab and in clinical settings to eliminate cancer from its root-the CSCs.

Another advantage of the disclosed methods of making microcapsules is the ability to encapsulate cells within an aqueous liquid. For example, pluripotent stem cells can be encapsulated in an aqueous liquid, such as a cellulose solution, such as a carboxymethyl cellulose solution, and cultured to maintain pluripotency.

The disclosed microcapsules can further contain one or more bioactive agents within the core, shell, or combination thereof. In some embodiments, the bioactive agent is a therapeutic agent. In some embodiments, the bioactive agent can be a biomolecule. In certain embodiments, the bioactive agent can be a differentiation agent, such as a growth factor or chemokine suitable to promote the growth, survival, pluripotency, or differentiation of the cells encapsulated within the microcapsules. In certain embodiments, the bioactive agent is a growth factor such as VEGF (vascular endothelial growth factor), FGF (fibroblast growth factor), TGF (transforming growth factor), or combinations thereof. In some embodiments, the bioactive agent is a therapeutic agent such as an immunosuppressant and/or an anti-inflammatory agent.

In some embodiments, the encapsulated stem cells are treated with one or more differentiation agents to produce an encapsulated pre-differentiated stem cell. Pre-differentiation helps to prevent teratoma formation. For example, the encapsulated stem cells can be treated with one or more of BMP-4 and bFGF to direct the stem cells toward the mesodermal-early cardiac lineage before transplantation. Embryonic stem cells can be treated with EGF and bFGF to induce the differentiation to neural progenitor cells before implantation. Implanted progenitor cells can be differentiated to astrocytes, oligodendrocytes, and mature neurons. This can be used therapeutically for neural disorder treatments or spinal cord injuries. Mesenchymal stem cells can be cultured in specialized medium with TGF-β to induce chondrogenic differentiation for cartilage repair. Mesenchymal stem cells can be induced with growth factor IGF-2 and BMP-9 to induce osteogenic differentiation for bone regeneration.

The encapsulated stem cells, with or without pre-differentiation, are in some embodiments released from the microcapsules prior to implantation, e.g., to mimic the physiologic process of the release of blastocyst from the zona pellucida for further differentiation. In some embodiments, the released stem cell aggregates are encapsulated in a biocompatible, biodegradable micro-matrix. The micro-matrix can be formed from a polyelectrolyte complex comprising one or more polycations and one or more polyanions. The micro-matrix can be formed throughout and/or surrounding the cell aggregates by sequential incubation of the cell aggregate in solutions of one or more polycations and one or more polyanions.

Suitable polyanions and polycations can be selected in view of a number of factors, including the desired in vivo stability of the micro-matrix (e.g., the desired in vivo biodegradation rate). Examples of suitable polycations include, for example, polypeptides, such as polyarginine, polylysine, polyhistidine, and polyornithine, polysaccharides, such as DEAE-dextran, chitosan, as well as synthetic polymers, such as polyallyamine or salts or quaternized derivatives thereof (e.g., polyallylamine hydrochloride), polyethyleneimine (PEI; e.g., linear PEI, branched PEI, or combinations thereof), modified derivatives of the above and mixtures thereof. Examples of suitable polyanions include, for example, polypeptides such as polyglutamic acid, polysaccharides, including alginates (e.g., sodium alginate), celluloses (e.g., cellulose sulfate), hyaluronic acid, and glycosaminoglycans such as chondroitin, proteins, such as heparin, as well as synthetic polymers, such as polystyrene sulfonate, modified derivatives of the above and mixtures thereof. In some embodiments, the polyelectrolyte complex can comprise one or more polyanions and one or more polycations selected from alginate, collagen, fibrin, hyaluronan, heparin, chondroitin, poly-l-lysine, ploy-l-glutamic acid, polyallylamine hydrochloride, polystyrene sulfonate, modified derivatives of the above and mixtures thereof.

For example, released cell aggregates can be encapsulated in a micro-matrix formed by soaking the aggregates in chitosan (e.g., 0.4% w/v) and then in oxidized alginate (e.g., 0.15% w/v) (or non-oxidized if slow degradation is desired) solution, optionally repeated one or more times. In preferred embodiments, the micro-matrix does not substantially increase the size of the stem cell aggregates.

These aggregates can also be encapsulated using the disclosed nonplanar microfluidic and coaxial electrospray methods.

The disclosed microcapsules can have an average diameter ranging from about 50 microns to about 1000 microns, including about 100 microns to about 500 microns. Another advantage of the disclosed microcapsules is the ability of the shell to have an average thickness greater than 10, 20, 30, 40, or 50 microns. For example, the shell can have an average thickness ranging from about 10 microns to about 100 microns, including about 50 microns.

Another advantage of the disclosed methods of making microcapsules is the ability to produce a core-shell microcapsule in a single step. Existing methods involve the use of coating steps, which can require the use of polyionic materials. Therefore, disclosed is a microcapsule comprising a core comprising a mammalian cell or cell aggregate suspended or encapsulated in a matrix; and a shell surrounding the core comprising a biocompatible hydrogel, wherein the matrix and biocompatible hydrogel are formed from materials having the same chemical composition, and wherein a polyionic material having a charge opposite from the charge of the material forming the matrix and the biocompatible hydrogel is not present between the core and the shell.

Another advantage of the disclosed methods and devices is the ability to produce microcapsules having controlled dimensions. For example, disclosed are microcapsules that are monodisperse in size. In some embodiments, microcapsules are produced with average diameters with less than 10%, 15%, 20%, 25%, or 30% variation.

Microencapsulated cells produced by the disclosed methods may be used for miniaturized 3D culture and differentiation of the cells, cryoprotection (e.g., cryopreservation), cell-based drug delivery, cell transplantation, tissue regeneration, and assisted reproductive medicine.

Microfluidic Devices

Provided are microfluidic devices configured to prepare the core-shell microcapsules described herein.

The microfluidic devices can comprise a core inlet channel, a first shell inlet channel, a second shell inlet channel, a first crosslinker inlet channel, and a second crosslinker inlet channel, all of which fluidly converge to form a flow focusing chamber; and an outlet channel flowing from the flow focusing chamber.

In some embodiments, the core inlet channel, first shell inlet channel, second shell inlet channel, first crosslinker inlet channel, and second crosslinker inlet channel can all converge at a single point to form a flow focusing chamber. In these embodiments, the flow focusing chamber can contain a single flow focusing region where fluid flowing from the core inlet channel, first shell inlet channel, second shell inlet channel, first crosslinker inlet channel, and second crosslinker inlet channel converge.

In other embodiments, the core inlet channel, first shell inlet channel, and second shell inlet channel can converge at an upstream region of the flow focusing chamber so as to form a first (upstream) flow focusing region where fluid flowing from the core inlet channel, first shell inlet channel, and second shell inlet channel converge, and the first crosslinker inlet channel and second crosslinker inlet channel can converge as a downstream region of the flow focusing chamber so as to form a second (downstream) flow focusing region where fluid flowing from the first (upstream) flow focusing region, first crosslinker inlet channel, and second crosslinker inlet channel converge.

The particular design of the microfluidic device, including the number and type of inlet channels with respect to the flow focusing chamber, the presence or absence of additional microfluidic components in the device, and the arrangement of the microfluidic components within the device, will be dependent upon a number of factors. These factors can include the intended composition of the microcapsules being formed by the microfluidic device, the desired microcapsule production rate, and the nature of the cells, cell aggregates, and/or bioactive agents that are incorporated within the microcapsules.

In some embodiments, the core inlet channel is positioned between the first shell inlet channel and the second shell inlet channel, such that the intersection of the core inlet channel and the flow focusing chamber is positioned in an intermediate location relative to the intersection of the first shell inlet channel and the flow focusing chamber and the intersection of the second shell inlet channel and the flow focusing chamber.

In some embodiments the core inlet channel, the first shell inlet channel, and the second shell inlet channel are positioned between the first crosslinker inlet channel and the second crosslinker inlet channel, such that the intersection of the core inlet channel and the flow focusing chamber, the intersection of the first shell inlet channel and the flow focusing chamber, and the intersection of the second shell inlet channel and the flow focusing chamber are all positioned in an intermediate location relative to the intersection of the first crosslinker inlet channel and the flow focusing chamber and the intersection of the second crosslinker inlet channel and the flow focusing chamber.

In certain embodiments, the distance between the core inlet channel and the first shell inlet channel is substantially equal (e.g., within 15%, within 10%, within 5%, within 2.5%, or within 1%) to the distance between the core inlet channel and the second shell inlet channel. Similarly, in some embodiments, the distance between the core inlet channel and the first crosslinker inlet channel is substantially equal (e.g., within 15%, within 10%, within 5%, within 2.5%, or within 1%) to the distance between the core inlet channel and the second crosslinker inlet channel.

In some instances, the distance between the core inlet channel and the first crosslinker inlet channel, and the distance between the core inlet channel and the second crosslinker inlet channel is less than about 15 times the width of the core inlet channel (e.g., less than about 10 times the width, less than about 7 times the width, less than about 5 times the width, or less than about 3 times the width).

In some embodiments, the first crosslinker inlet channel and the second crosslinker inlet channel are configured such that fluid flow from the first crosslinker inlet channel into the flow focusing chamber and fluid flow from the second crosslinker inlet channel into the flow focusing chamber is substantially perpendicular (e.g., within about ±10 degrees of perpendicular, within about ±7 degrees of perpendicular, within ±5 degrees of perpendicular, or within ±3 degrees of perpendicular) to fluid flow from the core inlet channel into the flow focusing chamber.

In some embodiments, the first shell inlet channel and second shell inlet channel configured such that fluid flow from the first shell inlet channel into the flow focusing chamber and fluid flow from the second shell inlet channel into the flow focusing chamber is substantially parallel (e.g., within about ±10 degrees of parallel, within about ±7 degrees of parallel, within ±5 degrees of parallel, or within ±3 degrees of parallel) to fluid flow from the core inlet channel into the flow focusing chamber. In other embodiments, the first shell inlet channel and second shell inlet channel configured such that fluid flow from the first shell inlet channel into the flow focusing chamber and fluid flow from the second shell inlet channel into the flow focusing chamber is substantially perpendicular (e.g., within about ±10 degrees of perpendicular, within about ±7 degrees of perpendicular, within ±5 degrees of perpendicular, or within ±3 degrees of perpendicular) to fluid flow from the core inlet channel into the flow focusing chamber.

The dimensions of the microfluidic channels in the device (e.g., the core inlet channel, shell inlet channels, crosslinker channels, and outlet channel) can individually and/or in combination be selected in view of a number of factors, including the cells and/or cellular aggregates to be encapsulated, the presence or absence of cells and/or cellular aggregates in the microcapsule shell, the desired microcapsule size, the desired ratio of core to shell material with the microcapsule, and combinations thereof.

The core inlet channel has a width and height that are large enough to permit passage of the cell or cell aggregate that is being encapsulated within the microcapsule core. In some embodiments, the core inlet channel has a width that ranges from about 50 microns to about 300 microns (e.g., from about 50 microns to about 275 microns, from about 75 microns to about 250 microns, from about 75 microns to about 200 microns, from about 75 microns to about 175 microns, or from about 100 microns to about 150 microns). In some embodiments, the core inlet channel has a height that ranges from about 50 microns to about 300 microns (e.g., from about 50 microns to about 275 microns, from about 75 microns to about 250 microns, from about 75 microns to about 200 microns, from about 75 microns to about 175 microns, or from about 100 microns to about 150 microns).

In some embodiments, first shell inlet channel and the second shell inlet channel have substantially equivalent cross-sectional dimensions, meaning that the height and width of the first shell inlet channel are substantially equivalent (e.g., within 10%, within 5%, within 3%, within 2%, or within 1%) to the height and width of the second shell inlet channel).

In some embodiments, the width of the first shell inlet channel and the width of the second shell inlet channel are between about 15% and about 95% (e.g., between about 20% and about 90%, between about 30% and about 80%, or between about 40% and about 70%) of the width of the core inlet channel. In certain embodiments, the width of the first shell inlet channel and the width of the second shell inlet channel are between about 10 microns and about 200 microns (e.g., between about 15 microns and about 175 microns, between about 20 microns about 150 microns, between about 20 microns and about 125 microns, or between about 25 microns and about 100 microns).

In some embodiments, the height of the first shell inlet channel and the height of the second shell inlet channel are between about 100% and about 300% (e.g., between about 125% and about 275%, between about 150% and about 250%, or between about 175% and about 225%) of the height of the core inlet channel. In certain embodiments, the height of the first shell inlet channel and the height of the second shell inlet channel are between about 100 microns and about 400 microns (e.g., between about 125 microns and about 350 microns, between about 125 microns about 300 microns, between about 150 microns and about 275 microns, or between about 150 microns and about 250 microns).

In some embodiments, first crosslinker inlet channel and the second crosslinker inlet channel have substantially equivalent cross-sectional dimensions, meaning that the height and width of the first crosslinker inlet channel are substantially equivalent (e.g., within 10%, within 5%, within 3%, within 2%, or within 1%) to the height and width of the second crosslinker inlet channel).

In some embodiments, the width of the first crosslinker inlet channel and the width of the second crosslinker inlet channel are between about 15% and about 600% (e.g., between about 50% and about 500%, between about 100% and about 400%, or between about 150% and about 300%) of the width of the core inlet channel. In certain embodiments, the width of the first crosslinker inlet channel and the width of the second crosslinker inlet channel are between about 10 microns and about 600 microns (e.g., between about 75 microns and about 550 microns, between about 150 microns about 500 microns, between about 250 microns and about 450 microns, or between about 350 microns and about 400 microns).

In some embodiments, the height of the first crosslinker inlet channel and the height of the second crosslinker inlet channel are between about 15% and about 500% (e.g., between about 100% and about 400%, between about 150% and about 350%, or between about 200% and about 300%) of the height of the core inlet channel. In certain embodiments, the height of the first crosslinker inlet channel and the height of the second crosslinker inlet channel are between about 10 microns and about 600 microns (e.g., between about 50 microns and about 550 microns, between about 100 microns about 500 microns, between about 200 microns and about 450 microns, or between about 300 microns and about 400 microns).

As described above, in some embodiments, the core inlet channel, first shell inlet channel, second shell inlet channel, first crosslinker inlet channel, and second crosslinker inlet channel can all converge at a single point to form a flow focusing chamber. In these embodiments, the core inlet channel, first shell inlet channel, and second shell inlet channel can be fluidly connected to the flow focusing chamber immediately upstream of the first crosslinker inlet channel and the second crosslinker inlet channel.

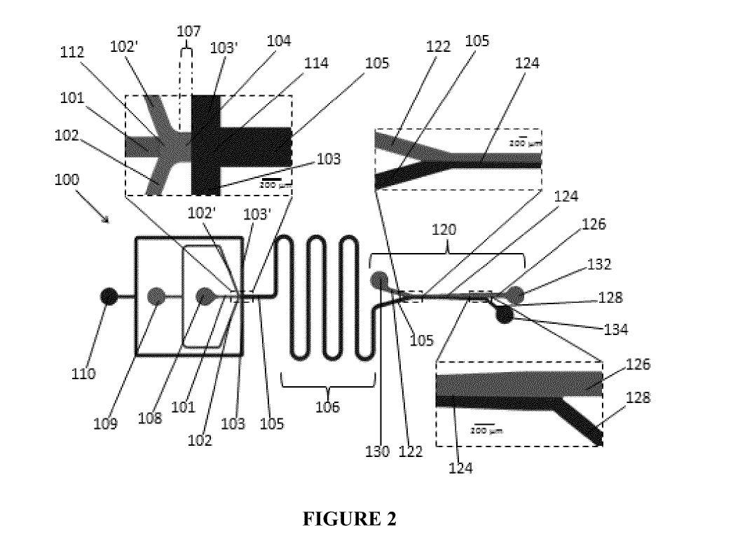

In other embodiments, the core inlet channel, first shell inlet channel, and second shell inlet channel can converge at an upstream region of the flow focusing chamber so as to form a first (upstream) flow focusing region where fluid flowing from the core inlet channel, first shell inlet channel, and second shell inlet channel converge, and the first crosslinker inlet channel and second crosslinker inlet channel can converge at a downstream region of the flow focusing chamber so as to form a second (downstream) flow focusing region where fluid flowing from the first (upstream) flow focusing region, first crosslinker inlet channel, and second crosslinker inlet channel converge. In some of these embodiments, the core inlet channel, first shell inlet channel, and second shell inlet channel can converge some distance upstream from where the first crosslinker inlet channel and second crosslinker inlet channel converge. For example, the core inlet channel, first shell inlet channel, and second shell inlet channel can converge at a distance upstream from where the first crosslinker inlet channel and second crosslinker inlet channel converge (see FIG. 2, 107) of from about 10 microns and about 600 microns (e.g., from about 75 microns and about 500 microns, from about 100 microns about 500 microns, from about 100 microns and about 450 microns, or from about 100 microns and about 300 microns).

The dimensions of the outlet channel can also be varied in view of the overall device architecture and dimensions of the microcapsules being prepared. In some embodiments, the outlet channel has a width that ranges from the width of the core inlet channel to the combined width of the core inlet channel, the first and second shell inlet channels, and first and second crosslinker inlet channels. In some embodiments, the height of the outlet channel is substantially equal to the height of the core inlet channel, the height of the first and/or second shell inlet channel, the height of the first and/or second crosslinker inlet channel, or combinations thereof.

The outlet channel can have any suitable length. In some instances, the outlet channel comprises and elongated and/or circuitous incubation region which is configured to increase the flow time of microcapsules within the microfluidic device. This can provide time for appropriate cross-linking of the microcapsule prior to the microcapsule leaving the outlet channel.

The outlet channel can comprise a separator region. The separator region can be configured to transfer microcapsules formed using the microfluidic device from an organic (oil) phase to an aqueous phase. The separator region can comprise a separator inlet channel that converges with the outlet channel to form a separation channel. The separation channel can then diverge downstream, forming an aqueous fluid outlet channel and an organic fluid outlet channel. The separator inlet channel can be fluidly connected to a separator fluid reservoir. The aqueous fluid outlet channel and the organic fluid outlet channel can be fluidly connected to an aqueous fluid reservoir and an organic fluid reservoir respectively.

The separator region can be configured to transfer microcapsules formed using the microfluidic device from an organic (oil) phase to an aqueous phase. For example, an organic phase comprising microcapsules can flow into the separation channel from the outlet channel, and an aqueous phase can flow into the separation channel from the separator inlet channel. The separation channel can be configured to provide for laminar flow of the aqueous phase and the organic phase through the separation channel. As the microcapsules flow through the separation channel, they can be extracted from the organic phase into the aqueous phase, and flow from the separation channel into the aqueous fluid outlet channel.

The separation channel can have a length selected to provide for extraction of the microcapsules formed using the microfluidic device from the organic (oil) phase flowing through the separation channel to the aqueous phase flowing through the separation channel. An appropriate separation channel length can be selected in view of a number of factors, including the identity of the organic phase and the aqueous phase, the chemical properties of the microcapsules (e.g., microcapsule hydrophobicity/hydrophilicity), and the physical properties of the microcapsules (e.g., microcapsule elasticity). In some embodiments, the separation channel can have a length of from about 1 mm to about 15 mm (e.g., from about 1 mm to about 10 mm, or from about 3 mm to about 7 mm).

The separator region can further include a directing element. The directing element can be any suitable device component configured to apply a force to microcapsules flowing through the separation channel. For example, an organic phase comprising microcapsules can flow into the separation channel from the outlet channel, and an aqueous phase can flow into the separation channel from the separator inlet channel. The separation channel can be configured to provide for laminar flow of the aqueous phase and the organic phase through the separation channel. As the microcapsules flow through the separation channel, a directing element can apply a force to the microcapsules to direct them from the organic phase into the aqueous phase. Once in the aqueous phase, the microcapsules can flow from the separation channel into the aqueous fluid outlet channel.

For example, in some embodiments, the directing element can be a dielectrophoretic element configured to apply a non-uniform electric field to all or part of the separation channel. For example, the dielectrophoretic element can include a first dielectrophoretic channel and a second dielectrophoretic channel fluidly connected to the separation channel. A potential bias can be applied across the first dielectrophoretic channel and the second dielectrophoretic channel to apply a non-uniform electric field to a portion of the separation channel. The nature of the electric field (e.g., the frequency of the electric field) can be varied as desired, for example, to direct microcapsules from the organic phase into the aqueous phase. Because the strength of the force exerted on the microcapsules is dependent on a variety of factors including the electrical properties (e.g., the composition) of the microcapsules, by varying the nature of the electric field (e.g., the frequency of the electric field), microcapsules of a particular composition (e.g., microcapsules containing encapsulated cells or cell aggregates) can be selectively directed from the organic phase into the aqueous phase while microcapsules of another composition (e.g., microcapsules without encapsulated cells or cell aggregates) can continue to flow through the separation channel in the organic phase.

The microfluidic device can further comprise one or more fluid reservoirs. In some cases, the core inlet channel is fluidly connected to a core fluid reservoir. In some embodiments, the first shell inlet channel and the second shell inlet channel are fluidly connected to a shell fluid reservoir. In certain embodiments, the first and second shell inlet channels are fluidly connected to the same shell fluid reservoir. In some embodiments, the first crosslinker inlet channel and the crosslinker shell inlet channel are fluidly connected to a crosslinker fluid reservoir. In certain embodiments, the first and second crosslinker inlet channels are fluidly connected to the same crosslinker fluid reservoir.

The microfluidic device can further comprise a dispatching channel fluidly connected to the core inlet channel. The dispatching channel can be configured to control the flow of mammalian cells or cell aggregates through the core inlet channel by providing a flow of fluid into the core inlet channel along its fluid flow path toward the flow focusing chamber, such that one large (e.g., 50-150 μm) cell (e.g., a single oocytes or embryo) or cell aggregate (e.g., a single ovarian follicle) is encapsulated in each microcapsule formed in the flow focusing chamber. A dispatching channel fluid reservoir can be fluidly connected to the dispatching channel.

The microfluidic device can further include one or more additional components (e.g., pressure gauges, valves, gaskets, pressure inlets, pumps, computer-controlled solenoid valves, fluid reservoirs, and combinations thereof) to facilitate device function and microcapsule formation.

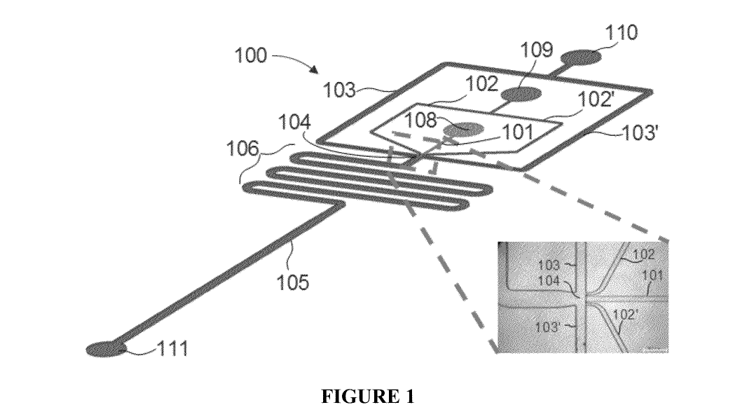

An example microfluidic device (100) is illustrated in FIG. 1. The microfluidic device (100) comprises a core inlet channel (101), a first shell inlet channel (102), a second shell inlet channel (102′), a first crosslinker inlet channel (103), and a second crosslinker inlet channel (103′), all of which fluidly converge to form a flow focusing chamber (104). The flow focusing chamber (104) contains a single flow focusing region where fluid flowing from the core inlet channel (101), the first shell inlet channel (102), the second shell inlet channel (102′), the first crosslinker inlet channel (103), and the second crosslinker inlet channel (103′) converge. The device further comprises an outlet channel (105) flowing from the flow focusing chamber (104).

The core inlet channel (101) is positioned between the first shell inlet channel (102) and the second shell inlet channel (102′), such that the intersection of the core inlet channel (101) and the flow focusing chamber (104) is positioned in an intermediate location relative to the intersection of the first shell inlet channel (102) and the flow focusing chamber (104) and the intersection of the second shell inlet channel (102′) and the flow focusing chamber (104).

The core inlet channel (101), the first shell inlet channel (102), and the second shell inlet channel (102′) are positioned between the first crosslinker inlet channel (103) and the second crosslinker inlet channel (103), such that the intersection of the core inlet channel (101) and the flow focusing chamber (104), the intersection of the first shell inlet channel (102) and the flow focusing chamber (104), and the intersection of the second shell inlet channel (102′) and the flow focusing chamber (104) are all positioned in an intermediate location relative to the intersection of the first crosslinker inlet channel (103) and the flow focusing chamber (104) and the intersection of the second crosslinker inlet channel (103′) and the flow focusing chamber (104).

The microfluidic device further comprises multiple fluid reservoirs. The upstream end of the core inlet channel (101) is fluidly connected to a core fluid reservoir (108). The upstream ends of the first and second shell inlet channels (102 and 102′) are fluidly connected to a shell fluid reservoir (109). The upstream ends of the first and second crosslinker inlet channels (103 and 103′) are fluidly connected to a crosslinker fluid reservoir (110).

The microfluidic device also includes an outlet channel (105). The outlet channel (105) comprises and elongated and/or circuitous incubation region (106) which is configured to increase the flow time of microcapsules within the microfluidic device. The downstream end of the outlet channel (105) is fluidly connected to an outlet fluid reservoir (111).

In some embodiments, the core inlet channel, first shell inlet channel, and second shell inlet channel can converge at an upstream region of the flow focusing chamber so as to form a first (upstream) flow focusing region where fluid flowing from the core inlet channel, first shell inlet channel, and second shell inlet channel converge, and the first crosslinker inlet channel and second crosslinker inlet channel can converge at a downstream region of the flow focusing chamber so as to form a second (downstream) flow focusing region where fluid flowing from the first (upstream) flow focusing region, first crosslinker inlet channel, and second crosslinker inlet channel converge.

An example microfluidic device (100) comprising a flow focusing chamber having a first flow focusing region and a second flow focusing region is illustrated in FIG. 2. The microfluidic device (100) comprises a core inlet channel (101), a first shell inlet channel (102), a second shell inlet channel (102′), a first crosslinker inlet channel (103), and a second crosslinker inlet channel (103′), all of which fluidly converge to form a flow focusing chamber (104). In this case, the core inlet channel (101), first shell inlet channel (102), and second shell inlet channel (102′) converge at an upstream region of the flow focusing chamber (112) so as to form a first (upstream) flow focusing region where fluid flowing from the core inlet channel (101), first shell inlet channel (102), and second shell inlet channel (102′) converge. The first crosslinker inlet channel (103) and the second crosslinker inlet channel (103′) converge at a downstream region of the flow focusing chamber (114) so as to form a second (downstream) flow focusing region where fluid flowing from the first (upstream) flow focusing region, first crosslinker inlet channel (103), and second crosslinker inlet channel (103′) converge. The core inlet channel (101), first shell inlet channel (102), and second shell inlet channel (102′) converge at a distance (107, approximately 150 microns) upstream from where the first crosslinker inlet channel (103) and the second crosslinker inlet channel (103′) converge. The device further comprises an outlet channel (105) flowing from the flow focusing chamber (104).

The core inlet channel (101) is positioned between the first shell inlet channel (102) and the second shell inlet channel (102′), such that the intersection of the core inlet channel (101) and the flow focusing chamber (104) is positioned in an intermediate location relative to the intersection of the first shell inlet channel (102) and the flow focusing chamber (104) and the intersection of the second shell inlet channel (102′) and the flow focusing chamber (104).

The core inlet channel (101), the first shell inlet channel (102), and the second shell inlet channel (102′) are positioned between the first crosslinker inlet channel (103) and the second crosslinker inlet channel (103), such that the intersection of the core inlet channel (101) and the flow focusing chamber (104), the intersection of the first shell inlet channel (102) and the flow focusing chamber (104), and the intersection of the second shell inlet channel (102′) and the flow focusing chamber (104) are all positioned in an intermediate location relative to the intersection of the first crosslinker inlet channel (103) and the flow focusing chamber (104) and the intersection of the second crosslinker inlet channel (103′) and the flow focusing chamber (104).

The microfluidic device further comprises multiple fluid reservoirs. The upstream end of the core inlet channel (101) is fluidly connected to a core fluid reservoir (108). The upstream ends of the first and second shell inlet channels (102 and 102′) are fluidly connected to a shell fluid reservoir (109). The upstream ends of the first and second crosslinker inlet channels (103 and 103′) are fluidly connected to a crosslinker fluid reservoir (110).

The microfluidic device also includes an outlet channel (105). The outlet channel (105) comprises and elongated and/or circuitous incubation region (106) which is configured to increase the flow time of microcapsules within the microfluidic device. The downstream end of the outlet channel (105) also comprises a separator region (120) configured to transfer microcapsules formed using the microfluidic device from an organic (oil) phase to an aqueous phase. The separator region (120) can comprise a separator inlet channel (122) that converges with the outlet channel (105) to form a separation channel (124). The separation channel (124) diverges downstream, forming an aqueous fluid outlet channel (126) and an organic fluid outlet channel (128). The upstream end of the separator inlet channel (122) is fluidly connected to a separator fluid reservoir (130). The downstream ends of the aqueous fluid outlet channel (126) and the organic fluid outlet channel (128) are fluidly connected to an aqueous fluid reservoir (132) and an organic fluid reservoir (134) respectively.

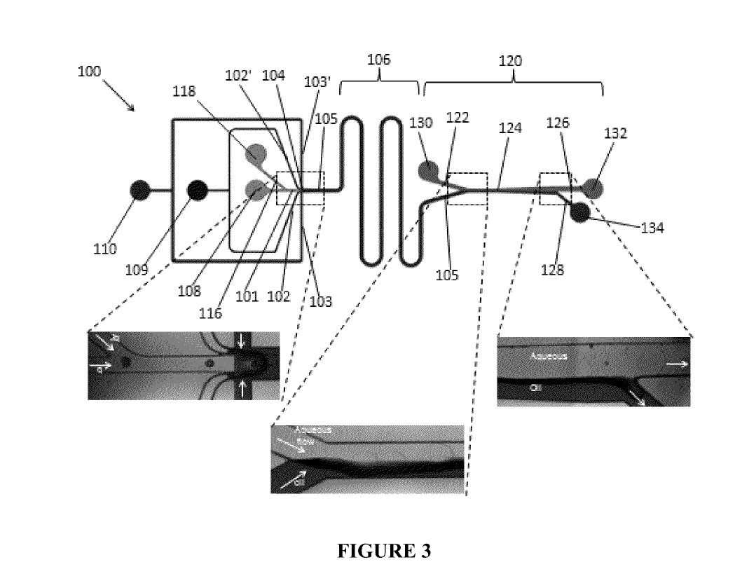

An example microfluidic device (100) comprising a dispatching channel and a separator region is illustrated in FIG. 3. The microfluidic device (100) comprises a core inlet channel (101), a first shell inlet channel (102), a second shell inlet channel (102′), a first crosslinker inlet channel (103), and a second crosslinker inlet channel (103′), all of which fluidly converge to form a flow focusing chamber (104). The flow focusing chamber (104) contains a single flow focusing region where fluid flowing from the core inlet channel (101), the first shell inlet channel (102), the second shell inlet channel (102′), the first crosslinker inlet channel (103), and the second crosslinker inlet channel (103′) converge. The device further comprises an outlet channel (105) flowing from the flow focusing chamber (104).

The core inlet channel (101) is positioned between the first shell inlet channel (102) and the second shell inlet channel (102′), such that the intersection of the core inlet channel (101) and the flow focusing chamber (104) is positioned in an intermediate location relative to the intersection of the first shell inlet channel (102) and the flow focusing chamber (104) and the intersection of the second shell inlet channel (102′) and the flow focusing chamber (104).

The core inlet channel (101), the first shell inlet channel (102), and the second shell inlet channel (102′) are positioned between the first crosslinker inlet channel (103) and the second crosslinker inlet channel (103), such that the intersection of the core inlet channel (101) and the flow focusing chamber (104), the intersection of the first shell inlet channel (102) and the flow focusing chamber (104), and the intersection of the second shell inlet channel (102′) and the flow focusing chamber (104) are all positioned in an intermediate location relative to the intersection of the first crosslinker inlet channel (103) and the flow focusing chamber (104) and the intersection of the second crosslinker inlet channel (103′) and the flow focusing chamber (104).

The microfluidic device (100) further includes a dispatching channel (116) fluidly connected to the core inlet channel (101) configured to control the flow of mammalian cells or cell aggregates through the core inlet channel (101) by providing a flow of fluid into the core inlet channel (101) along its fluid flow path toward the flow focusing chamber (104), such that one large cell or cell aggregate is encapsulated in each microcapsule formed in the flow focusing chamber (104). A dispatching channel fluid reservoir (118) is fluidly connected to the upstream end of the dispatching channel (116).

The microfluidic device (100) further comprises multiple fluid reservoirs. The upstream end of the core inlet channel (101) is fluidly connected to a core fluid reservoir (108). The upstream ends of the first and second shell inlet channels (102 and 102′) are fluidly connected to a shell fluid reservoir (109). The upstream ends of the first and second crosslinker inlet channels (103 and 103′) are fluidly connected to a crosslinker fluid reservoir (110).

The microfluidic device also includes an outlet channel (105). The outlet channel (105) comprises and elongated and/or circuitous incubation region (106) which is configured to increase the flow time of microcapsules within the microfluidic device. The downstream end of the outlet channel (105) also comprises a separator region (120) configured to transfer microcapsules formed using the microfluidic device from an organic (oil) phase to an aqueous phase. The separator region (120) can comprise a separator inlet channel (122) that converges with the outlet channel (105) to form a separation channel (124). The separation channel (124) diverges downstream, forming an aqueous fluid outlet channel (126) and an organic fluid outlet channel (128). The upstream end of the separator inlet channel (122) is fluidly connected to a separator fluid reservoir (130). The downstream ends of the aqueous fluid outlet channel (126) and the organic fluid outlet channel (128) are fluidly connected to an aqueous fluid reservoir (132) and an organic fluid reservoir (134) respectively.

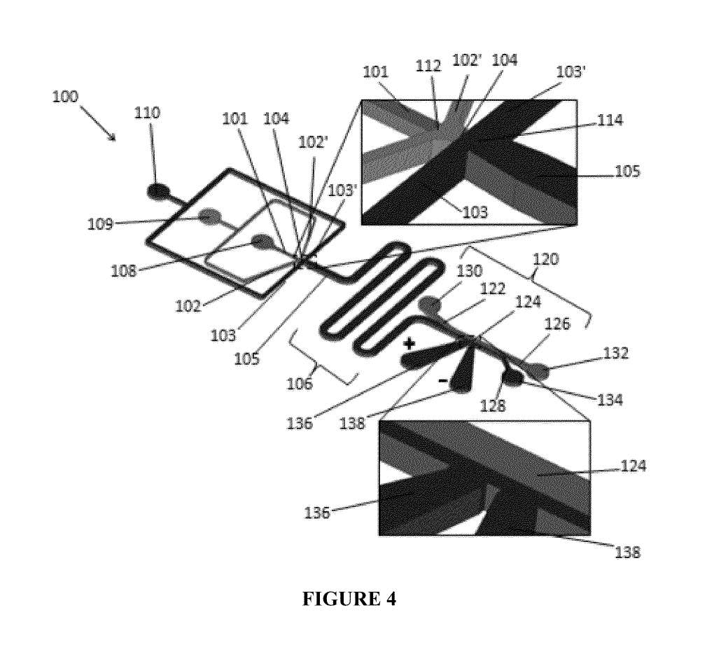

An example microfluidic device (100) comprising a separator region that further includes a directing element is illustrated in FIG. 4. The microfluidic device (100) comprises a core inlet channel (101), a first shell inlet channel (102), a second shell inlet channel (102′), a first crosslinker inlet channel (103), and a second crosslinker inlet channel (103′), all of which fluidly converge to form a flow focusing chamber (104). In this embodiment, the core inlet channel (101), first shell inlet channel (102), and second shell inlet channel (102′) converge at an upstream region of the flow focusing chamber (112) so as to form a first (upstream) flow focusing region where fluid flowing from the core inlet channel (101), first shell inlet channel (102), and second shell inlet channel (102′) converge. The first crosslinker inlet channel (103) and the second crosslinker inlet channel (103′) converge at a downstream region of the flow focusing chamber (114) so as to form a second (downstream) flow focusing region where fluid flowing from the first (upstream) flow focusing region, first crosslinker inlet channel (103), and second crosslinker inlet channel (103′) converge. The device further comprises an outlet channel (105) flowing from the flow focusing chamber (104).

The core inlet channel (101) is positioned between the first shell inlet channel (102) and the second shell inlet channel (102′), such that the intersection of the core inlet channel (101) and the flow focusing chamber (104) is positioned in an intermediate location relative to the intersection of the first shell inlet channel (102) and the flow focusing chamber (104) and the intersection of the second shell inlet channel (102′) and the flow focusing chamber (104).

The core inlet channel (101), the first shell inlet channel (102), and the second shell inlet channel (102′) are positioned between the first crosslinker inlet channel (103) and the second crosslinker inlet channel (103), such that the intersection of the core inlet channel (101) and the flow focusing chamber (104), the intersection of the first shell inlet channel (102) and the flow focusing chamber (104), and the intersection of the second shell inlet channel (102′) and the flow focusing chamber (104) are all positioned in an intermediate location relative to the intersection of the first crosslinker inlet channel (103) and the flow focusing chamber (104) and the intersection of the second crosslinker inlet channel (103′) and the flow focusing chamber (104).

The microfluidic device further comprises multiple fluid reservoirs. The upstream end of the core inlet channel (101) is fluidly connected to a core fluid reservoir (108). The upstream ends of the first and second shell inlet channels (102 and 102′) are fluidly connected to a shell fluid reservoir (109). The upstream ends of the first and second crosslinker inlet channels (103 and 103′) are fluidly connected to a crosslinker fluid reservoir (110).

The microfluidic device also includes an outlet channel (105). The outlet channel (105) comprises and elongated and/or circuitous incubation region (106) which is configured to increase the flow time of microcapsules within the microfluidic device. The downstream end of the outlet channel (105) also comprises a separator region (120) configured to transfer microcapsules formed using the microfluidic device from an organic (oil) phase to an aqueous phase. The separator region (120) comprises a separator inlet channel (122) that converges with the outlet channel (105) to form a separation channel (124). The separation channel (124) diverges downstream, forming an aqueous fluid outlet channel (126) and an organic fluid outlet channel (128). The upstream end of the separator inlet channel (122) is fluidly connected to a separator fluid reservoir (130). The downstream ends of the aqueous fluid outlet channel (126) and the organic fluid outlet channel (128) are fluidly connected to an aqueous fluid reservoir (132) and an organic fluid reservoir (134) respectively.

The separator region (120) further includes a directing element configured to apply a force to microcapsules flowing through the separation channel (124). By way of example, the separator region (120) further includes a dielectrophoretic element configured to apply a non-uniform electric field to all or part of the separation channel (124) that includes a first dielectrophoretic channel (136) and a second dielectrophoretic channel (138) fluidly connected to the separation channel (124). A potential bias can be applied across the first dielectrophoretic channel (136) and the second dielectrophoretic channel (138) to apply a non-uniform electric field to a portion of the separation channel (124). As described above, an organic phase comprising microcapsules can flow into the separation channel (124) from the outlet channel (105), and an aqueous phase can flow into the separation channel (124) from the separator inlet channel (122). The separation channel (124) can be configured to provide for laminar flow of the aqueous phase and the organic phase through the separation channel. As the microcapsules flow through the separation channel (124), non-uniform electric field provided by the dielectrophoretic element can exert a force on the microcapsules to direct them from the organic phase into the aqueous phase. Once in the aqueous phase, the microcapsules can flow from the separation channel (124) into the aqueous fluid outlet channel (126).

Methods of Making

The microfluidic devices described herein can be composed of any material suitable for the flow of fluid through the channels. For example, in some embodiments, the device is fabricated from a material that is chemically resistant to solvents used to prepare the microcapsules described herein (e.g., the material will not significantly dissolve in or react with the solvents over the timescale in which microcapsules are fabricated).

In some embodiments, the device is fabricated, in whole or in part, from glass, silicon, or combinations thereof. In some embodiments, the device is fabricated, in whole or in part, from a metal and/or metal alloys (e.g., iron, titanium, aluminum, gold, platinum, chromium, molybdenum, zirconium, silver, niobium, alloys thereof, etc.). In some embodiments, the device is fabricated, in whole or in part, from a polymer and/or plastic, including, but not limited to, polyesters, polycarbonate, polyethylene terephthalate (PET) polyethylene terephthalic ester (PETE), polytetrafluoroethylene (PTFE), polymethyl methacrylate (PMMA), polydimethylsiloxane (PDMS), polyurethane, bakelite, polyester, etc. The device can also be fabricated, in whole or in part, from a ceramic (e.g., silicon nitride, silicon carbide, titania, alumina, silica, etc.).

In certain embodiments, the device is fabricated, in whole or in part, from a photocurable epoxy. In certain embodiments, the device is fabricated, in whole or in part, from polydimethylsiloxane.

The devices can be fabricated using a variety of suitable methods known in the art. For example, the devices described herein can be formed by, for example, lithography, etching, embossing, or molding of a polymeric surface. In general, the fabrication process may involve one or more of any of the processes described below (or similar processes), and different parts of a device may be fabricated using different methods, and subsequently assembled or bonded together to form the final microfluidic device. Suitable fabrication methods can be selected in view of a number of factors, including the nature of the substrate(s) used to form the device as well as the dimensions of the microfluidic features making up the device.

Lithography involves use of light or other form of energy such as electron beam to selectively alter a substrate material. Typically, a polymeric material or precursor (e.g., photoresist, a lightresistant material) is coated on a substrate and is selectively exposed to light or other form of energy. Depending on the photoresist, exposed regions of the photoresist either remain or are dissolved in subsequent processing steps known generally as “developing.” This process results in a pattern of the photoresist on the substrate. In some embodiments, the photoresist is used as a master in a molding process. In some embodiments, a polymeric precursor is poured on the substrate with photoresist, polymerized (i.e., cured) and peeled off. The resulting polymer is bonded or glued to another flat substrate after drilling holes for inlets and outlets.

In some embodiments, the photoresist is used as a mask for an etching process. For example, after patterning photoresist on a silicon substrate, channels can be etched into the substrate using a deep reactive ion etch (DRIE) process or other chemical etching process known in the art (e.g., plasma etch, KOH etch, HF etch, etc.). The photoresist can then be removed, and the substrate can be bonded to another substrate using one of any bonding procedures known in the art (e.g., anodic bonding, adhesive bonding, direct bonding, eutectic bonding, etc.). Multiple lithographic and etching steps and machining steps such as drilling may be included as required.

In some embodiments, a polymeric substrate, such as PMMA, can be heated and pressed against a master mold for an embossing process. The master mold can be formed by a variety of processes, including lithography and machining. The polymeric substrate can then be bonded with another substrate to form the final device. Machining processes may be included if necessary.

Devices can also be fabricated using an injection molding process. In an injection molding process, a molten polymer or metal or alloy is injected into a suitable mold and allowed to cool and solidify. The mold typically consists of two parts that allow the molded component to be removed. Parts thus manufactured may be bonded to result in the device.

In some embodiments, sacrificial etch may be used to form the device. Lithographic techniques can be used to pattern a material on a substrate. This material can then be covered by another material of different chemical nature. This material can undergo lithography and etch processes, or another suitable machining process. The substrate can then be exposed to a chemical agent that selectively removes the first material. In this way, channels can be formed in the second material, leaving voids where the first material was present before the etch process.

In some embodiments, microchannels can be directly machined into a substrate by laser machining or CNC machining. If desired, several layers thus machined can be bonded together to obtain the final device.

Methods of Using

Microfluidic devices can be used to form the microcapsules described herein.

Microcapsules can be formed by simultaneously flowing an aqueous solution comprising cells or cell aggregates suspended in a matrix through the core inlet channel of the microfluidic device; flowing an aqueous solution comprising biocompatible hydrogel-forming material through the first shell inlet channel and the second shell inlet channel of the microfluidic device; and flowing an organic solution, dispersion, or emulsion comprising a crosslinking agent through the first crosslinker inlet channel and the second crosslinker inlet channel of the microfluidic device.

The solutions which flow into the microfluidic device to form the microcapsules are referred to as component solutions. The suspension flowed through the core inlet channel forms the microcapsule core. Cells and cell aggregates present in this suspension, as well as the liquid matrix in which the cells and/or cell aggregates are suspended, forms the microcapsule shell. Accordingly, additional materials, such as bioactive agents (e.g., small molecule therapeutics, biomolecules, nanoparticles and microparticles for drug delivery, and combinations thereof), can be incorporated into the microcapsule shell by including them in the suspension flowed through the core inlet channel. Similarly, the solution flowed through the shell inlet channels forms the microcapsule shell. Materials in addition to the biocompatible hydrogel, such as, for example, cells, cell aggregates, bioactive agents, and combinations thereof, can be incorporated into the microcapsule shell by including them in the solution flowed through the shell inlet channels.

The fluid streams from the core inlet channel, the first shell inlet channel, the second shell inlet channel, the first crosslinker inlet channel, and the second crosslinker inlet channel converge at the flow focusing chamber. The dimension and orientation of the channels, flow focusing chamber, and the fluid flow rates are configured such that a microcapsule precursor is formed within the flow focusing chamber. The microcapsule precursor comprises a cell or cell aggregate suspended in a liquid matrix surrounded by an aqueous solution comprising a biocompatible hydrogel-forming material. The microcapsule precursor is suspended in an organic solution, dispersion, or emulsion comprising a crosslinking agent. The microcapsule precursor flows from the flow focusing chamber and through the outlet channel. During this time, the crosslinking agent can react to crosslink the microcapsule precursor.

The crosslinking agent can be selected in view of the identity of the biocompatible hydrogel-forming material. For example, the crosslinking agent can be a divalent metal ion, such as calcium ions, barium ions, or combinations thereof, which can react to crosslink alginate. Other suitable crosslinking agents include, for example genipin, thrombin, polycationic polymers such as polylysine, and combinations thereof. In some embodiments, further crosslinking can be performed, for example, using UV irradiation.

In this way, the microcapsules described herein can be formed in a continuous process, as long as the flow of the component solutions is maintained. The flow rates of the component solutions can be varied to selected characteristics of the microcapsules.

For example, in one embodiment, the flow rate of the organic solution, dispersion, or emulsion comprising a crosslinking agent through the first crosslinker inlet channel and the second crosslinker inlet channel of the device can be adjusted to vary the average diameter of the microcapsules. In some embodiments, the flow rates through the first shell inlet channel, the second shell inlet channel, the core inlet channel, or combinations thereof can be adjusted to vary the ratio of core to shell materials in the microcapsules.

In certain embodiments, the fluid flow rate through the first and second shell inlet channels ranges from about 50 microliters/hour to about 200 microliters/hour (e.g., from about 75 microliters/hour to 175 microliters/hour, or from about 100 microliters/hour to 150 microliters/hour).

In certain embodiments, the fluid flow rate through the core inlet channel ranges from about 50 microliters/hour to about 150 microliters/hour (e.g., from about 65 microliters/hour to about 135 microliters/hour, or from about 75 microliters/hour to about 125 microliters/hour).

In certain embodiments, the fluid flow rate through the first and second crosslinker inlet channels ranges from about 2 millileters/hour to 10 millileters/hour (e.g., from about 3 microliters/hour to about 9 microliters/hour, from about 4 microliters/hour to 8 microliters/hour, or from about 5 microliters/hour to 7 microliters/hour).

Suitable flow rates can be adjusted, as required, for operation of a microfluidic device having a particular architecture.

Electrospray Devices

The core-shell microcapsules described herein can be made using an electrospray apparatus configured to prepare the core-shell microcapsules described herein. With reference now to FIG. 8, microcapsules can be made using an electrospray apparatus (200) having a coaxial needle (202) comprising a central lumen (204) surrounded by an outer lumen (206), a gelling bath comprising a crosslinking agent (208); and a power source (210) configure to provide a potential bias between the coaxial needle (202) and the gelling bath. The coaxial needle (202) can be configured such that fluids ejected from the coaxial needle (202) contact the gelling bath. The disclosed core-shell microcapsules can be made by simultaneously ejecting an aqueous solution comprising cells or cell aggregates suspended in a matrix (212) from the central lumen (204) and an aqueous solution comprising biocompatible hydrogel-forming material (214) from the outer lumen (206) into the gelling bath comprising a crosslinking agent (208). Simultaneous ejection can be achieved using syringes attached to syringe pumps under simultaneous control.

### EXAMPLES

### Example 1

### Microfluidic Flow Focusing Device

Materials and Methods

Fabrication of Microfluidic Device

A schematic of microfluidic flow focusing device to form core-shell microcapsules is shown in FIG. 1. The design of microfluidic channel comprised of a flow focusing chamber (inset in FIG. 1). Width and height of the core inlet channel, shell inlet channels, and crosslinker inlet channels at their point of intersection with the flow focusing chamber are 120×120 um, 50×220 um and 400×320 um, respectively. Total length of the device is 46 mm.

To fabricate polydimethyl siloxane (PDMS) microfluidic device, firstly, silicon master with patterned microfluidic channels were prepared using photolithographic technique. Briefly, photosensitive epoxy (SU-8 2025, Microchem) was spun coated onto the 4 inch wafer. The thickness of the first SU-8 coating was approximately 60 microns. The wafers were then soft baked at 95° C. for 9 min. The wafers were exposed to UV light through the first shadow mask (to pattern core channel), followed by the post exposure baking at 90° C. for 7 min. Two more layers of photoresist were spun coated at desired thickness and exposed with two different shadow masks (to pattern alginate shell and oil channel on the same silicon substrate). The SU-8 pattern on the substrate was developed in SU-8 developer (Microchem) for 10 min, rinsed with IPA (isopropyl alcohol) and then dried using nitrogen gas. PDMS was then poured on the silicon substrate and cured at 65° C. for 3 hours. Thereafter, PDMS was lifted off with the pattern on its surface. Two patterned PDMS layers was then treated with oxygen plasma for 30 seconds and then aligned and bonded together to produce final microfluidic device. Final device was then kept at 65° C. for 1 day before use for core-shell microcapsule formation.

Preparation of Reagents

Core of the microcapsules were composed of viscous carboxymethyl cellulose or collagen solution. 10 mg/ml solution of cellulose in 0.3 M Mannitol was prepared. 3-6 mg/ml neutral collagen solution was prepared as per the manufacture's protocol (BD Biosciences). Shell of the microcapsules was composed of 2% alginate solution prepared in 0.3 M Mannitol solution. All the solutions were buffered with 10 mM HEPES solution to maintain pH ˜7.2. In order to crosslink alginate in the microfluidic channel, calcium chloride was infused in mineral oil. Briefly, stable emulsion of mineral oil and calcium chloride solution (volume ratio-3:1) was prepared with the addition of 1.2% SPAN 80 as surfactant. Thereafter, water in the emulsion was evaporated using rotatory evaporator. Oil with calcium chloride solution remained stable and used as a continuous phase in the microfluidic channel for microcapsule formation.

Cell Culture

To maintain mouse (R1) embryonic stem cells in undifferentiated state, medium consisted of Knockout DMEM supplemented with 15% (v/v) Knockout Serum, 4 mM L-glutamine, 100 ug/ml antibiotics and 10³ U/ml leukemia inhibiting factor, 10 ug/ml gentamicin and 0.1 mM mercaptoethanol. Cells were passaged when they reached desired confluence. To encapsulate cells in core-shell microcapsules, cells were detached using trypsin and gently pipetted to break aggregates. Cells were counted and mixed with the above-mentioned carboxymethyl cellulose or collagen solution at 5 million/ml cell density.

Encapsulation of Embryonic Stem Cells in Core-Shell Microcapsules

Syringe pumps (Harvard Apparatus) were employed for the injection of fluids into the microfluidic system. Microfluidic device was linked to syringes via poly(tetrafluoroethylene) (PTFE) and silicone tubings. Aqueous (cellulous or collagen) solution with cells, alginate, and oil were injected into the device through inner, middle and outer channel of the microfluidic device. Flow rates of the liquids were optimized to form microcapsules of different sizes. Microcapsules were collected in a tube containing medium. Tube was then centrifuged at 300 rpm to separate microcapsules from oil to aqueous phase. All the solutions were kept at 4° C. to lower the metabolic activity of cells during the encapsulation process. Live/dead assay was performed to check the viability of cells after the encapsulation process.

Results

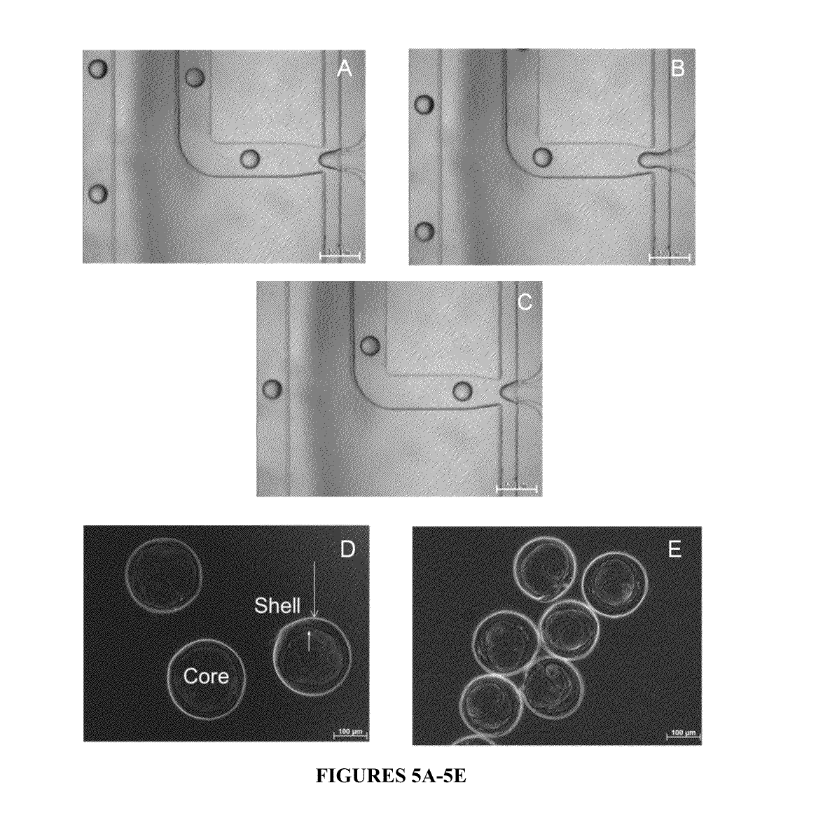

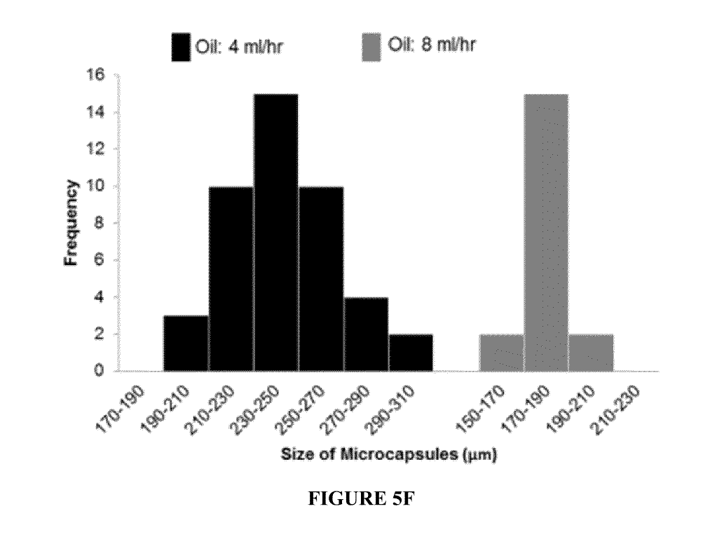

FIG. 1 is a schematic of a microfluidic device. A core microfluidic inlet channel 101, and two shell microfluidic inlet channels 102 and 102′, and two crosslinker microfluidic inlet channels 103 and 103′, were used to inject aqueous solution with cells, aqueous alginate solution, and mineral oil infused with calcium, respectively. The process of microcapsule formation in this microfluidic device is shown in FIGS. 5A-5C. Phase contrast images (10×) showing microcapsules of various sizes could be obtained by varying the flow rates of core, shell, and oil solutions are shown in FIGS. 5D and 5E. 250 μm microcapsules were obtained using flow rates 90, 200 and 4000 μl/hr (FIG. 5D). 175 μm microcapsules were obtained using flow rates, 90, 200 and 8000 μl/hr. FIG. 5F is a histogram depicting size distribution of microcapsules at two different flow rates. At low oil flow rate, 242 μm mean microcapsules (SD 23 μm) were obtained, whereas at higher flow rate, 181.1 μm mean microcapsules (SD 10.6 μm) were obtained. Flow rates are reported in order for core, shell and oil flow respectively.

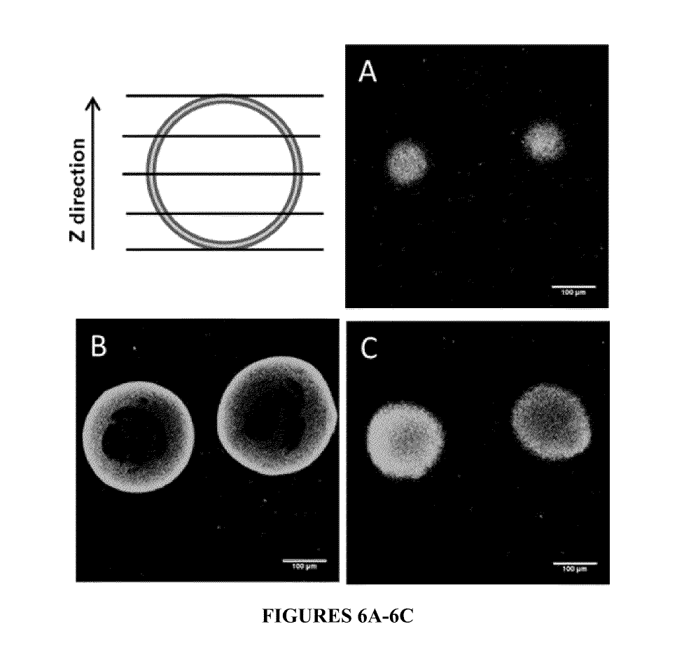

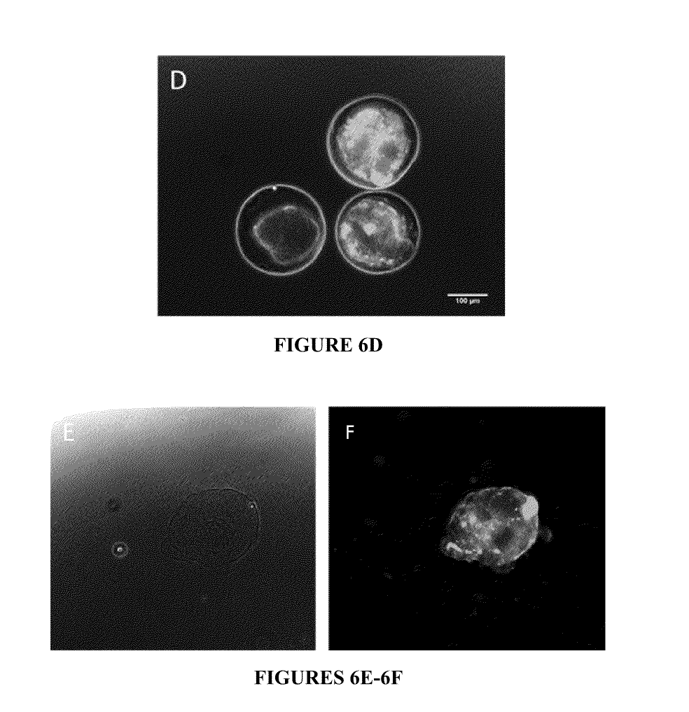

FIGS. 6A-6C are confocal images of microcapsules stained with FITC IgG, showing the core-shell structure of the microcapsule. Thickness of the microcapsule shell was observed to be approximately 50 μm. FIG. 6D is a phase contrast image of collagen core microcapsules with collagen stained with FITC. FIGS. 6E and 6F show the phase contrast and fluorescent images of the collagen gel in the microcapsule core after alginate shell of the microcapsule is removed using sodium citrate.

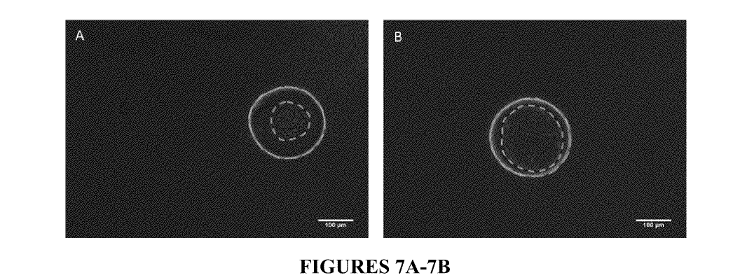

The size of the core (or thickness of the shell) can be controlled by regulating the core fluid flow rates (FIG. 7). FIG. 7A shows a microcapsule with a small core obtained at low core flow rate, whereas FIG. 7B shows a microcapsule with a large core obtained at a high flow rate.

The microfluidic device was used to encapsulate R1 embryonic stem (ES) cells in carboxymethyl cellulose core-alginate shell microcapsules. Phase contrast (FIGS. 8A-8B) and fluorescent (FIG. 8C) images of the encapsulated ES cells were obtained at day 0. These cells had a viability right after encapsulation greater than 92% (FIG. 8C). At day 3, images of the encapsulated ES cells show the formation of multiple ES cell aggregates (FIGS. 8D-8E). By day 9, the multiple cell aggregates have merged to form a single embryoid body (EB) cell aggregate in each microcapsule (FIGS. 8G-8H), indicating the microcapsules may be used to control the aggregate size.

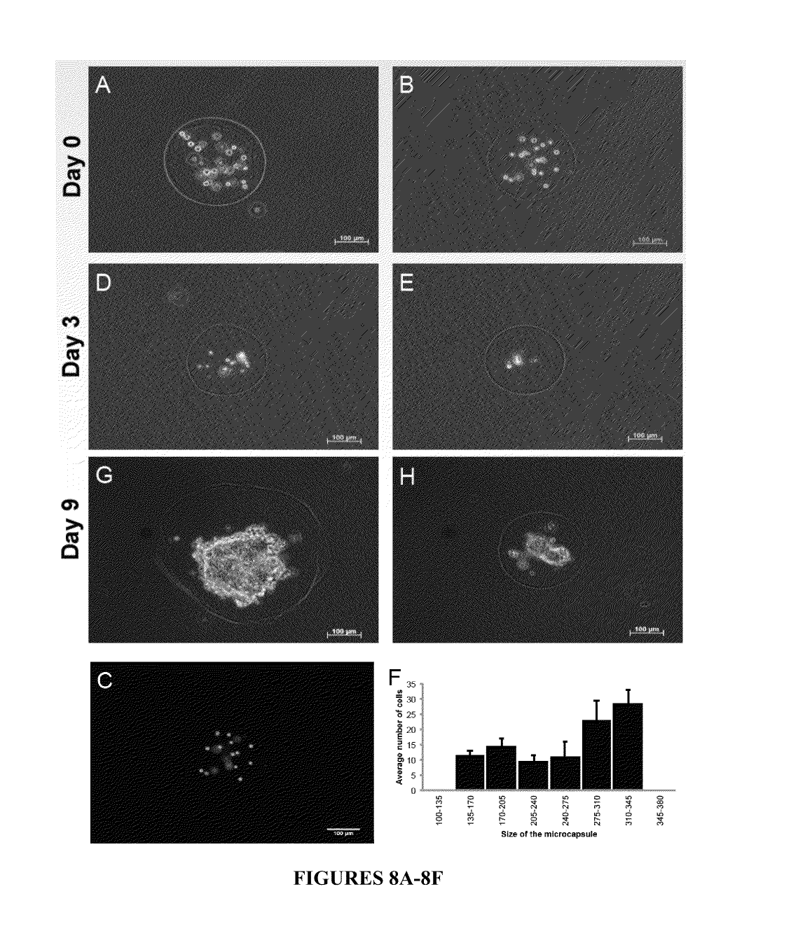

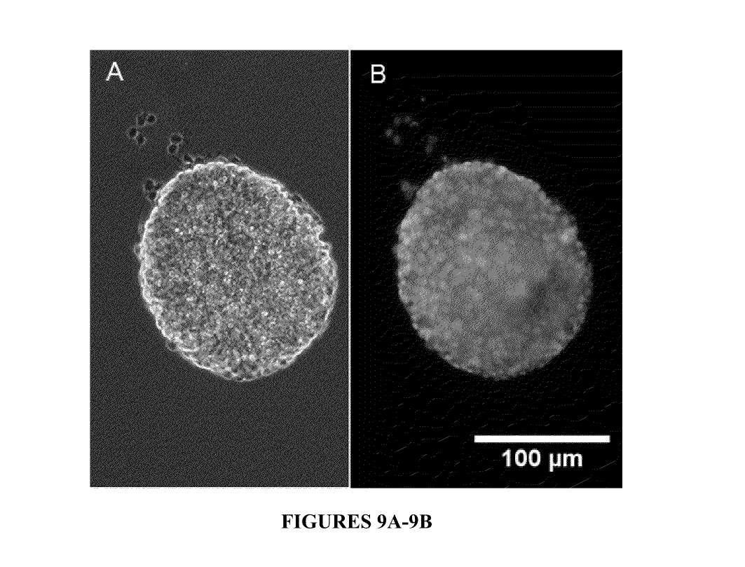

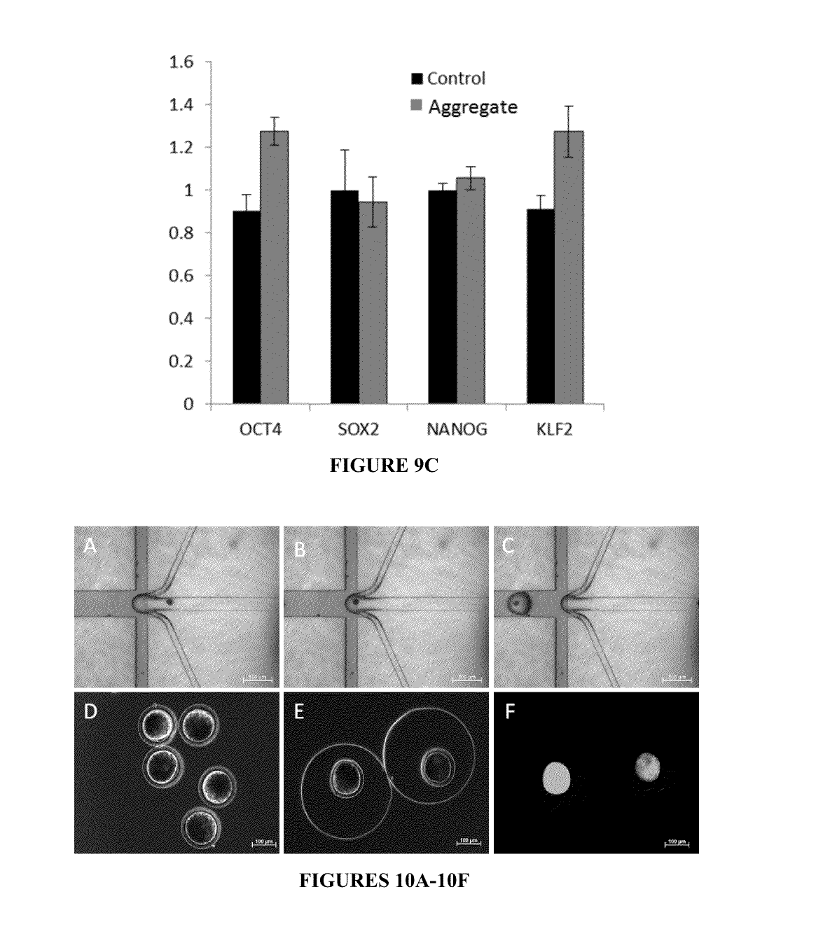

Pluripotency of the ES cell aggregates at day 9 was confirmed using OCT4 staining (FIGS. 9A-9B). Real time reverse transcription (RT) PCR was also used to compare expression of Oct4, SOX2, NANOG, and KLF2 pluripotency genes between control ES cells and microencapsulated ES cell aggregates (FIG. 9C).

The microfluidic device was also used to microencapsulate porcine oocytes. FIGS. 10A-10C are time lapse images showing this encapsulation. FIGS. 10D-10E are micrograph showing non-encapsulated porcine oocyte (FIG. 10D) and oocytes encapsulated in alginate microcapsules (FIG. 10E). As shown in FIG. 10F, the microencapsulated oocytes are alive.

### Example 2

### Coaxial Electrospray Device

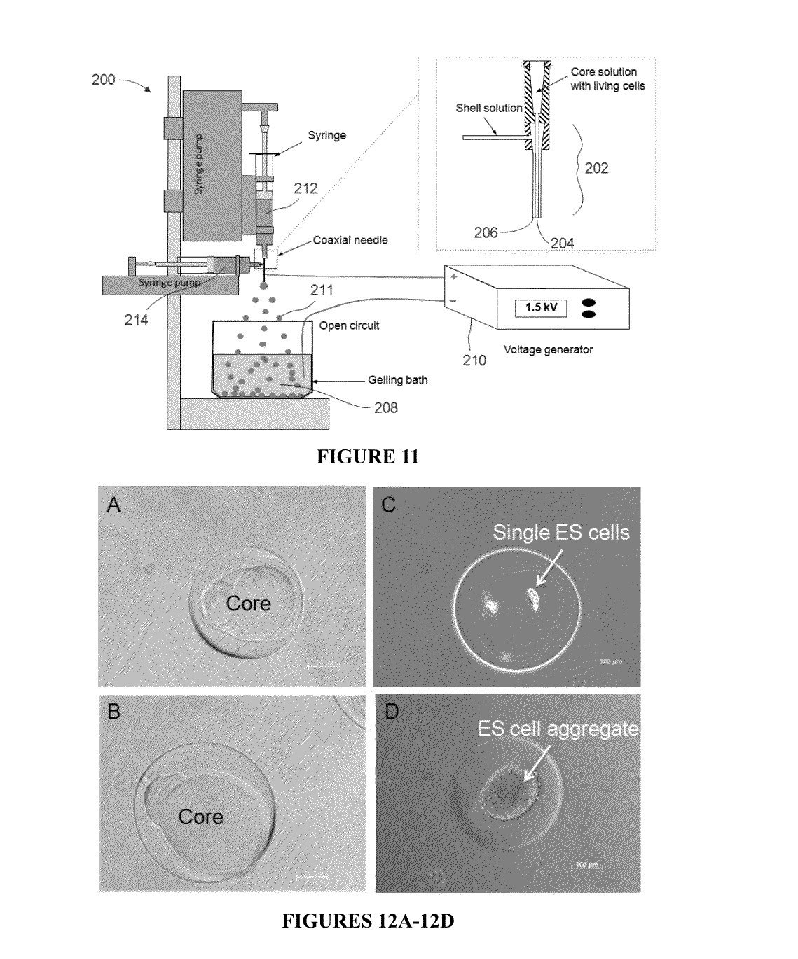

A coaxial electrospray device for preparing core-shell microcapsules was produced. FIG. 11 is a schematic of the coaxial electrospray setup. A core solution suspended with cells and alginate shell solution are first pumped into the inner and outer lumens of a custom-made coaxial needle, respectively. The two solutions are then sprayed under a low electrostatic field into a collection bath of calcium chloride solution to crosslink alginate in the microcapsule outer shell. The zoom-in view is presented to illustrate the design of the coaxial needle.

Detached cells in physiological saline were centrifuged and re-suspended at a density of 5×10⁶ cells/ml in 10 mg/ml carboxymethylcellulose sodium salt (sigma, 50% high viscosity and 50% medium viscosity) with 0.25 M mannitol as the core solution which was pumped into the inner lumen of coaxial needle at 30 μl/min. The 2% alginate with 0.25 M mannitol was pumped into the outer lumen as the shell solution at 70 μl/min. The core and shell solutions were sprayed under an electrostatic field (1.2 KV to 1.8 kV depending on desired sizes) into 150 mM calcium chloride solution for gelling/crosslinking alginate to form hydrogel. Then, the core-shell microcapsules were collected, washed using 0.5 M mannitol solution, and finally suspended in culture medium. The custom made coaxial needle with 28 G inner lumen and 21 G outer lumen was custom-fabricated by Rame-Hart Instrument Co. (Succasunna, N.J.).

By tuning the voltage of electrostatic field and/or flow rates of the core and shell solutions, microcapsules of various (100-500 μm) but uniform size and shell thickness can be consistently fabricated. To illustrate, small microcapsules with 100 μm core and 200 μm overall size (FIG. 12A) and large ones with 300 μm core and 400 μm overall size (FIG. 12B) in diameter were fabricated in high and low voltage field, respectively. R1 mouse embryonic stem cells were also encapsulated in large microcapsules as single cells (FIG. 12C) that proliferated to form a single cell aggregate in each microcapsule after 7 days (FIG. 12D).

### Example 3

### Coaxial Electrospray of Microcapsules with an Aqueous Liquid Core and Alginate Hydrogel Shell for Encapsulation and Miniaturized 3D Culture of Pluripotent Stem Cells

Materials and Methods

Knockout® DMEM and serum for ES cell culture were purchased from Life Technologies. Leukemia inhibitory factor (LIF) was purchased from Millipore. Trypsin/EDTA, regular DMEM (high glucose), and the live/dead viability/cytotoxicity kit for mammalian cells were purchased from Invitrogen. Fetal bovine serum (FBS), penicillin, and streptomycin were purchased from Hyclone. Primary antibodies were purchased from Abcam. Secondary antibodies for immunohistochemistry and HRP-linked antibodies for western blotting were purchased from Invitrogen and Cell Signaling, respectively. Alginate was purchased from Sigma and purified by washing in chloroform with charcoal and dialyzing against deionized water, followed by freeze drying. All other chemicals were purchased from Sigma unless specifically indicated otherwise.

Cell Culture:

R1 murine ES cells from ATCC were cultured in ES cell medium made of Knockout® DMEM supplemented with 15% Knockout® serum, 1000 U/ml LIF, 4 mM/glutamine, 0.1 M 2-mercaptoethanol, 10 μg/ml gentamicin, 100 U/ml penicillin, and 100 μg/ml streptomycin in gelatin coated tissue culture flasks with medium being changed daily. For cardiac differentiation, ES cells were cultured in regular DMEM supplemented with 25 ng/ml BMP-4, 5 ng/ml bFGF, 100 U/ml penicillin, and 100 mg/l streptomycin for 3 days, followed by maintaining in regular DMEM supplemented with 20% FBS. All cells were cultured at 37° C. in a humidified 5% CO₂ incubator.

Coaxial Electrospray of Core-Shell Microcapsules:

The coaxial electrospray system and a cross-sectional view of the coaxial needle are illustrated in FIG. 11. The system includes two syringe pumps for pushing the core fluid with living cells and shell fluid of alginate through the concentric inner and outer lumens in the coaxial needle, respectively. Under an open electrostatic field from a voltage generator, concentric drops of the two coaxial fluids at the needle tip were broken up into microdrops and sprayed into the gelling bath containing 100 mM calcium chloride solution to instantly gel alginate in the shell fluid before the two fluids got mixed. The core fluid contained ES cells at 5×10⁶ per ml, 0.25 M aqueous mannitol, and either 2% sodium alginate (w/v) for making microcapsules with an alginate hydrogel core or 1% (w/v) sodium carboxymethyl cellulose for making microcapsules with a liquid core. The shell fluid consisted of 2% purified alginate (w/v) in 0.25 M aqueous mannitol solution. After encapsulation, the microcapsules with ES cells in the gelling solution were washed with 0.5 M mannitol solution for 7 min and suspended in ES cell medium after removing mannitol for further culture.

Cell Viability, Proliferation, and Pluripotency:

Viability of the encapsulated ES cells was determined using the live/dead assay kit. Proliferation of the encapsulated ES cells was monitored as the formation of cell aggregates in the microcapsule core. The pluripotency of aggregated ES cells on day 7 was examined by qRT-PCR for gene expression and immunoblotting and immunohistochemical staining at the level of protein expression. In brief, ES cell aggregates formed in the hydrogel or liquid core were released from microcapsules by incubating in 55 mM sodium citrate solution for 30 s and washing in 1×PBS for 3 min. RNAs in the released or 2D culture cell aggregates were isolated using an RNeasy Plus Mini Kit (Qiagen) following the manufacture's instruction. Synthesis of complementary DNAs (cDNAs) was performed using the iScript™ cDNA synthesis kit (Bio-Rad) according to the manufacture's instruction. A Bio-Rad CFX96 real time PCR machine was used to quantify gene expression. Relative gene expression was calculated using the ΔΔCₜ method built in the Bio-Rad software. The expression of five pluripotency genes including Oct-4, Sox-2, Nanog, Klf2, and SSEA-1 was studied. For the immunoblotting, a total of 0.2 ml ice-cold RIPA lysis buffer (Bio7 rad) with complete proteinase inhibitor cocktail (Roche) was added to the pelleted cell aggregates after centrifuging and pipetted vigorously. The lysate was then sonicated and stored at −20° C. On the second day, the lysate was thawed and centrifuged at 16,000 g for 20 min at 4° C. The total protein concentration was determined using a Bio-rad protein assay kit. A total of 20 μg total protein was then loaded to each lane of SDS-PAGE gel for electrophoresis with a Mini Protean Tetra System (Bio-rad) according to the manufacturer's instructions. Quantitative analysis of protein expression was done using ImageJ to quantify the total intensity over the area occupied by the protein bands obtained from immunoblotting. For immunohistochemical staining of Oct-4 and SSEA-1 to study pluripotency at the protein expression level, the aggregates from the aqueous liquid core were fixed with 4% paraformaldehyde for 15 min at room temperature and then incubated with 3% bovine serum albumin at room temperature for 30 min to block non-specific binding. The primary antibodies for Oct-4 and SSEA-1 were then applied by overnight incubation at 4° C. and the corresponding secondary antibodies were added for 1 h for fluorescence labeling at room temperature in the dark. The samples were further stained for nuclei by incubating with 5 μM Hoechst 33342 for 15 min before examination using an Olympus FV1000 confocal microscope.

Cardiac Differentiation:

After 7 days culture, the aggregates formed in hydrogel core as well as liquid core were released using 55 mM sodium citrate, plated in gelatin-coated dish, and cultured with differentiation medium containing BMP-4 and bFGF for 3 days, followed by maintaining in regular DMEM supplemented with 20% FBS for up to 2 weeks. For the 2D aggregate/colony differentiation, ES cells are plated on the gelatin-coated dish and cultured in ES complete growth medium with LIF for 3 days to form colonies/aggregates that were induced to differentiate using the same differentiation medium for 3 days, followed by maintaining in regular DMEM supplemented with 20%. The cumulative percentage of beating aggregates or colonies on each day out of the total number of aggregates plated was counted and calculated starting from the day of initiating differentiation. To stain cTnT for flow cytometry analysis, cells before and after differentiation (on day 14) were collected by trypsin and fixed using 4% paraformaldehyde for 15 min at 4° C. The fixed samples were then blocked using 5% BSA for 30 min and the differentiated cells were further incubated with cTnT antibody for 45 min at 4° C. in the dark. All the samples were then resuspended in 0.2 ml ice-cold PBS and studied using BD LSR-II flow cytometer. The data were further analyzed using FlowJo software. For gene expression study, RNAs of the cell aggregates/colonies before and after differentiation were obtained using the RNeasy Plus mini kit (Qiagen). The cDNAs were then synthesized for cTnT, Nkx2.5, and Brachyury or T genes. Immunohistochemical analyses were done by fixing the attached cell aggregates from the 3D liquid core on day 13 when the beating reaches the plateau, staining the fixed samples with antibodies targeting cTnT, α-actinin, and connexin 43 according to the manufacture's protocol, and examining with an Olympus FV1000 confocal microscope.

Statistical Analysis:

Student's two-tailed t-test assuming equal variance was calculated using Microsoft® Excel to determine statistical significance (p<0.05).

Results

The coaxial electrospray setup is illustrated in FIG. 11. The core and shell aqueous fluids were injected into the inner and outer lumen of a coaxial needle, respectively. Under an open electrostatic field, drops of the two fluids at the tip of the coaxial needle were broken up and sprayed into the gelling bath of 100 mM Ca²⁺ to instantly gel alginate in the shell fluid. In order to form a core-shell structure, mixing between the core and shell fluids was minimized before alginate is gelled, which was achieved by adding 1% sodium carboxymethyl cellulose in the core fluid to raise its viscosity. Cellulose, a major polysaccharide in plant cell wall, was chosen to be the viscosity modifier because of its non-toxic nature to mammalian cells. The high viscosity of both the cellulose-based core fluid and alginate-based shell fluid together with the fast gelling kinetics of alginate in calcium chloride solution provided for the formation of microcapsules with a liquid core and hydrogel shell. The core-shell morphology of the resultant microcapsules of various (˜250, 300, and 400 μm) but fairly uniform sizes was evident using microscopy. The alginate hydrogel shell was also evident by adding 1% FITC-labeled dextran (500 kD) in the shell fluid to make the microcapsules for examination using confocal fluorescence microscopy.

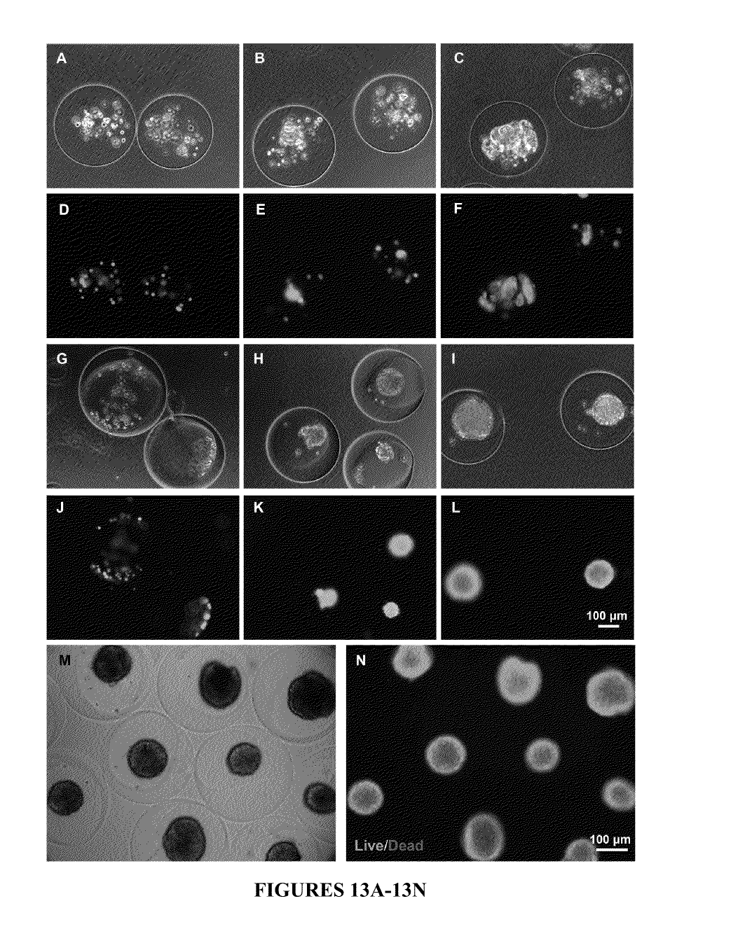

For cell microencapsulation, ES cells were suspended in the core fluid at a density of 5×10⁶ cells/ml and electrospray was done under the following conditions: core flow rate, 47 μl/min; shell flow rate, 90 μl/min, and voltage: 1.8 kV. The core fluid is either 2% sodium alginate or 1% carboxymethyl cellulose solution for making microcapsules with either a hydrogel or aqueous core, respectively. Typical morphology of the resultant microcapsules with an ES cell-laden hydrogel and liquid core on day 0, 3, and 7 is shown in FIGS. 13A-13C and FIGS. 13G-13I, respectively. The corresponding fluorescence images of ES cells in the hydrogel and liquid core are given in FIGS. 13D-13F and FIGS. 13J-13L, respectively. Approximately 50 ES cells were encapsulated in the (hydrogel or liquid) core of each microcapsule with high viability (>90%) on day 0, which indicates the mild nature of the coaxial electrospray process. The encapsulated cells in the liquid core proliferated and started to form multiple small aggregates on day 3 that eventually merged together to form one single aggregate of 128.9±17.4 μm in the liquid core of each microcapsule on day 7 as shown in FIGS. 13G-13L. However, ES cells in the hydrogel core form relatively smaller aggregates with many dead single cells on day 3 and eventually form multiple irregular aggregates in each microcapsule on day 7 as shown in FIG. 13A-13F. The non-uniform size and irregular shape of ES cell aggregates in the microcapsules with a hydrogel core are probably due to the cross-linked alginate fibers that prevent ES cells from continuously growing to merge into single aggregates, which would not occur in the liquid core that resembles the native physiological microenvironment of ES cells in a pre-hatching stage embryo consisting of an aqueous liquid core of embryonic cells. Typical images of a larger field showing more single ES cell aggregates with high cell viability in microcapsules with a liquid core on day 7 are shown in FIGS. 13M-13N. The high viability of cells during the extended culture in the microcapsules with a liquid core is probably due to the relatively more efficient transport of nutrients and oxygen to all cells in the liquid core as a result of the miniaturized dimension, compared to macrocapsules or bulk gel that have been used for 3D culture of cells.

The fact that only approximately 50 ES cells are sufficient to obtain a cell aggregate of approximately 130 μm on day 7 (more than one thousand ES cells are usually needed initially with the commonly used hanging drop method for ES cell culture) makes it particularly attractive for stem cell-based personalized medicine, for which the number of cells available are usually limited. Another major advantage of using the coaxial electrospray technology is its high efficiency for mass production of ES cell aggregates to meet the high demand for tissue regeneration in clinical applications, considering the fact that approximately 100,000 microcapsules can be produced in approximately 20 minute in one step and that many ES cell aggregates can be obtained within 7 days. Lastly and importantly, the proliferation of ES cells in the core enclosed by the alginate hydrogel shell resembles the proliferation of embryonic (including ES) cells in the core of a pre-hatching stage embryo encompassed by zona pellucida (the hydrogel shell of embryo) during embryo development from a few cells to a few tens of cells and later a single merged cell aggregate (i.e., blastocyst) in each embryo. Therefore, the core-shell microcapsule system generated in one step using coaxial electrospray should provide a biomimetic, miniaturized 3D liquid culture microenvironment for pluripotent stem cells to better maintain their pluripotency than the 3D hydrogel and conventional 2D culture methods to be discussed below.

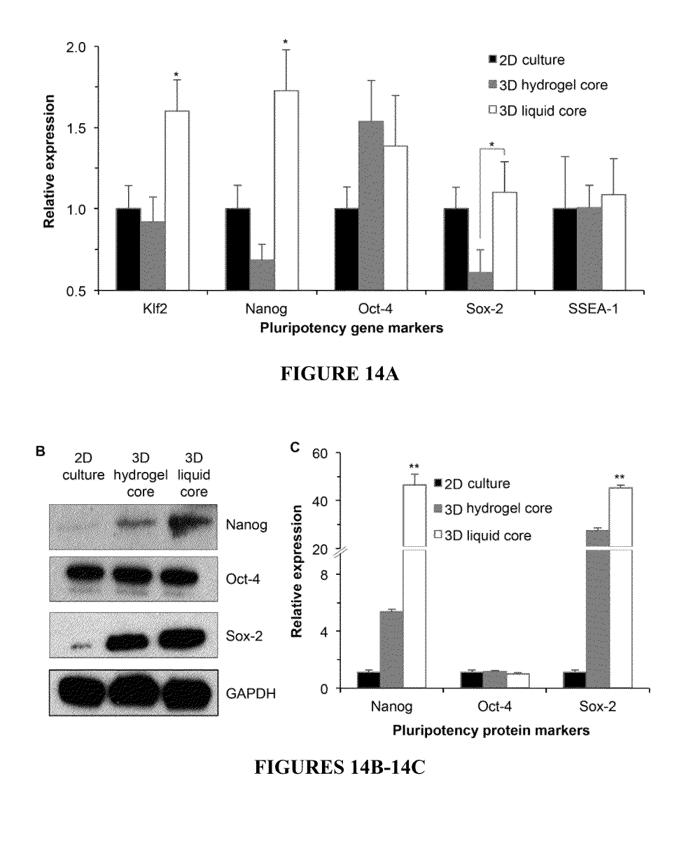

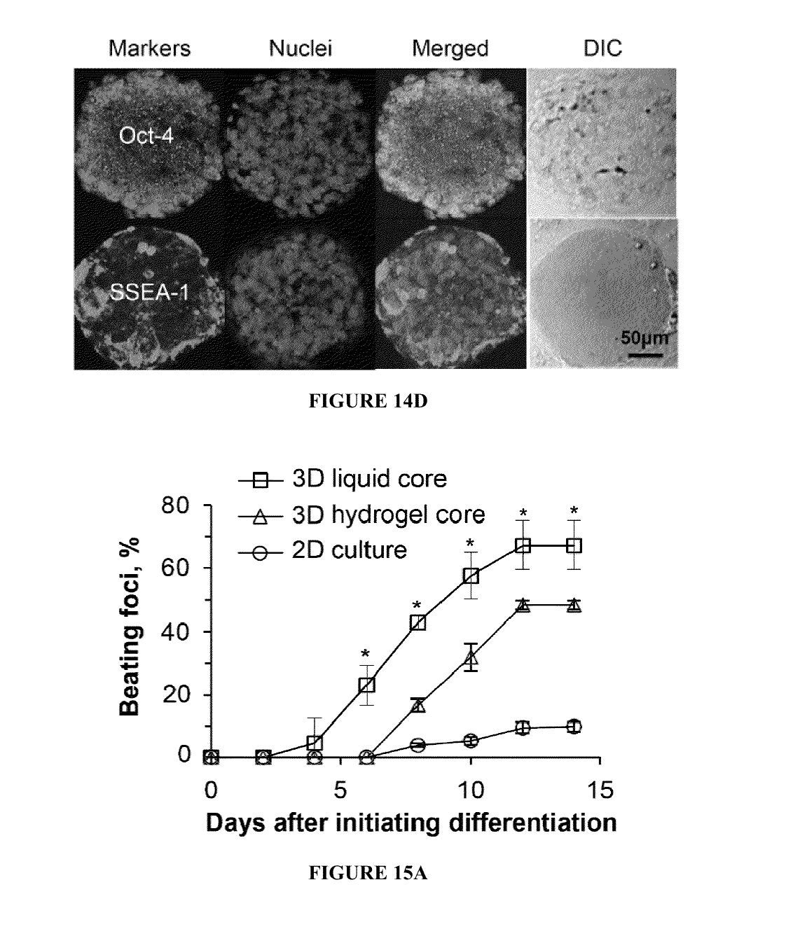

To investigate the pluripotency of aggregated ES cells on day 7, quantitative RT-PCR (qRT-PCR) analyses of 5 pluripotency marker genes including Klf2, Nanog, Oct-4, Sox-2, and SSEA-1 were conducted and the results are shown in FIG. 14A. The aggregated ES cells obtained by culturing in the miniaturized 3D liquid core have significantly higher expression of Nanog (1.73 times) and Klf2 (1.6 times) genes than that obtained by the conventional 2D culture, although the expression is not significantly different in terms of Oct-4, Sox-2, and SSEA-1 genes. Moreover, the ES cell aggregates formed in the 3D liquid core also had significantly higher expression of Klf2 (1.74 times), Nanog (2.55 times), and Sox-2 (1.83 times) than the cells cultured in 3D alginate hydrogel core. To further confirm the difference in pluripotency at the level of protein expression, immunoblotting studies were conducted on three representative pluripotency markers. The typical qualitative expression and quantitative analysis of three independent runs are shown in FIGS. 14B and 14C, respectively. ES cell aggregates formed in the 3D liquid core had significantly much higher expression of both Nanog (46.5 times) and Sox-2 (45.7 times) compared to the 2D cultured cells. The expression of Nanog (8.7 times) and Sox-2 (1.7 times) was also significantly higher in the ES cell aggregates obtained in 3D liquid than hydrogel core. It is not significantly different for the three groups in terms of the expression of Oct-4 protein, which is consistent with the gene expression data for Oct-4 in FIG. 3A. These data are not only consistent with what has been reported in recent years that gene and protein expression in cells cultured under 2D and 3D conditions could be very different but also indicate that a miniaturized 3D liquid microenvironment is crucial to culturing pluripotent stem cells. High pluripotency of the aggregated ES cells obtained in the miniaturized 3D liquid core is further confirmed at the level of protein expression by immunohistochemical staining of Oct-4 and SSEA-1 (FIG. 14D). Therefore, the biomimetic, miniaturized 3D culture in the pre-hatching stage embryo-like core-shell microcapsules indeed is beneficial for culturing ES cells to maintain high pluripotency.

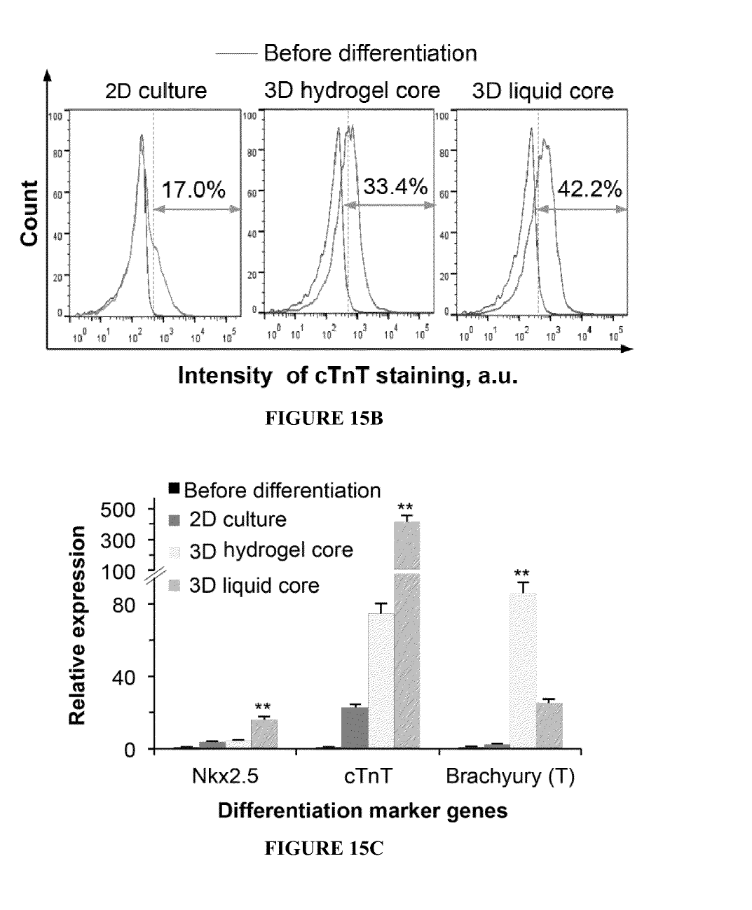

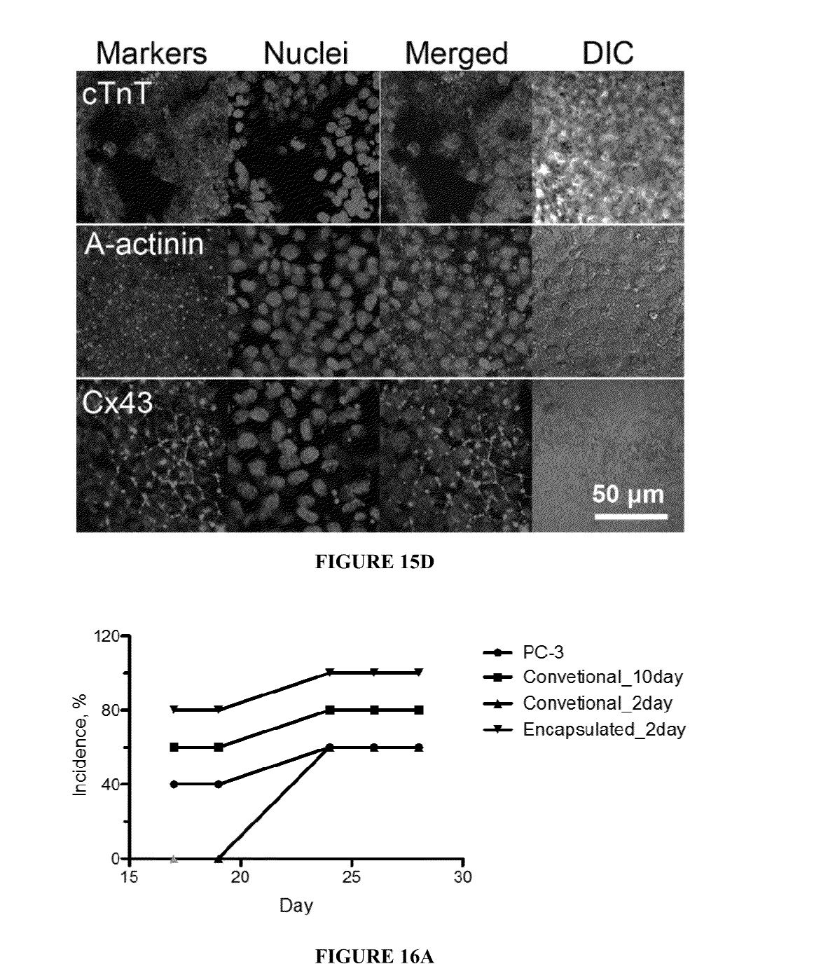

To further ascertain their high pluripotency, cardiac differentiation of the aggregated ES cells obtained after 7 days culture in the pre-hatching stage embryo-like core-shell microcapsules was induced using BMP-4 and bFGF for 3 days, followed by maintaining the cell aggregates in regular medium for up to 14 days. Both BMP-4 and bFGF have been used to induce mesoderm specification and cardiac commitment from stem cells. The successful differentiation into cardiac lineage was first confirmed by the cumulative percentage of beating foci as shown in FIG. 15A. For the aggregates obtained in the 3D liquid core, beating was first observed on day 4 (i.e., 4 days after initiating differentiation) and the cumulative percentage of beating aggregates reached the maximum of approximately 67.2% on day 12. Starting from day 6, the beating percentage for the differentiated aggregates from the 3D liquid core was significantly higher than that from 2D culture (9.75% maximum on day 12) and 3D hydrogel core (48.3% maximum on day 12). The morphology of cells in the beating aggregates suggested the presence of cardiomyocytes together with cardiac stromal cells such as fibroblasts, similar to the mixture of multiple types of cells naturally residing in the heart. The percentage of cardiomyocytes among the cells differentiated from ES cell aggregates in 3D liquid core was further estimated to be 42.2% based on flow cytometry analysis using cTnT as the marker protein of cardiomyocytes (FIG. 15B), which was higher than that differentiated from ES cell aggregates obtained from 2D culture (17.0%) and 3D hydrogel core (33.4%). Moreover, it was also close to the percentage of cardiomyocytes in native cardiac tissue. The differentiation of ES cell aggregates in a liquid core was further studied at the gene expression level using qRT-PCR for cardiac troponin T (cTnT), Nkx2.5, brachyury (or T) genes: the cTnT gene is specifically expressed in cardiomyocytes, Nkx2.5 is only present in the heart from the time of cardiac specification, and the brachyury (or T) gene is an early mesoderm marker. As shown in FIG. 15C, compared to the cells before differentiation, the expression of cTnT and Nkx2.5 in the differentiated cells was significantly (up to 414 and 16.3 times, respectively, for the 3D liquid core) higher, indicating successful direct differentiation of the ES cells into the cardiac lineage. Moreover, the expressions of these two cardiac markers were significantly higher in cells differentiated from ES cell aggregates from the 3D liquid core than that from 2D culture and 3D hydrogel core. The expression of the early mesoderm marker T protein in the cells differentiated from ES cell aggregates from the 3D liquid core was lower than that in the 3D alginate hydrogel core, which is probably because more of the former cells (from 3D liquid core) passed the early mesoderm stage and committed specifically into the cardiac lineage on day 14 (the day for the qRT-PCR study) than the latter ones (from 3D hydrogel core). In addition, immunohistochemical staining was performed to visualize the expression of cTnT, α-actinin, and connexin 43 (Cx43), proteins that are critical for cardiomyocytes to function (beat) properly. Both cTnT and α-actinin are key proteins in the sarcomere (the force generation unit) of cardiomyocytes and Cx43 is in the gap junction between cardiomyocytes that is crucial for synchronized beating in mature heart tissue. As shown in FIG. 15D, all these important proteins were highly expressed in the cells differentiated from ES cell aggregates obtained from the liquid core on day 13 of differentiation, which ultimately should promote the aggregates to beat. Therefore, the differentiated cell aggregates may closely mimic native cardiac tissue and may be transplanted directly to treat cardiac diseases such as myocardial infarction (MI). These results demonstrate that the disclosed coreshell microcapsules can be used as a biomimetic, miniaturized 3D system for culturing ES cells to maintain their pluripotency for further directed cardiac differentiation.

### Example 4

### Pre-Differentiation of ES Cell Aggregates and ACM Encapsulation of the Pre-Differentiated Cell Aggregates Using Chitosan and Oxidized Alginate

Ischemic heart disease related myocardial infarction (MI) is the leading cause of mortality in western countries and increasingly in the rest of the world. Damage during MI involves death of cardiomyocytes that poorly proliferate in the heart in response to injury, leading to decrease in cardiac output and eventual heart failure. While there are interventions available to reestablish coronary perfusion and treat arrhythmias associated with MI, there is no effective treatment available today to directly treat cell death from MI.

Cardiac tissue regeneration using stem cells to increase heart performance represents an important potential therapeutic option for treating MI. Some general challenges in using stem cells for cardiac regeneration are the dismal cell retention and viability of transplanted stem cells, as well as poor differentiation of the transplanted stem cells into cardiomyocytes after implantation.

Coaxial electrospray and microfluidic technologies were used to encapsulate mouse embryonic stem (ES) cells in the aqueous liquid core of microcapsules with an alginate hydrogel shell for miniaturized 3D culture to better maintain their pluripotency. The core-shell architecture of the microcapsules resembles that of the native home of ES cells, the pre-hatching embryos with an aqueous liquid-like core of embryonic cells and a hydrogel shell known as the zona pellucida. After forming aggregates in the microcapsules, the cells were treated with BMP-4 and bFGF to induce pre-differentiation into the cardiac lineage. Afterward, the aggregates were released out of the microcapsules to mimic the hatching step of early embryo development when blastocyst is released out of the zona pellucida (embryo shell) before further differentiation. The released aggregates were then encapsulated in a micro-matrix of oxidized alginate and chitosan (AC) that is biodegradable and biocompatible. The AC micro-matrix (ACM) encapsulated aggregates containing 2×10⁵ pre-differentiated ES cells were injected into the periphery of the infarct zone in mice subjected to permanent surgical occlusion of the left anterior descending (LAD) artery. The ACM encapsulated cells were found to increase survival time as well as cardiac structure and function compared to mice treated with saline or ACM only. Labeling the encapsulated cells showed the appearance of new cells that express multiple cardiomyocyte markers in the infarct zone. Remarkably, while injection of non-encapsulated cell aggregates results in formation of large tumors (87.5%), this was not observed with ACM encapsulated cell aggregates.

Using this strategy, the retention, viability, and differentiation of the injected cells into cardiac cells was improved while tumor formation after injection into the infarct zone of mouse hearts was minimized. These preliminary results indicate that the ACM encapsulation approach can improve the efficacy of direct injection of stem cells as a regenerative approach for the treatment of MI. Based on these results, it is hypothesized that ACM encapsulation of pre-differentiated stem cell aggregates allows differentiation into cardiac resident cells that can improve heart structure and function following MI without tumor formation.

Ischemic heart disease or myocardial infarction (MI) due to coronary arteriosclerosis remains the single largest cause of mortality in western countries and is increasingly common in other parts of the world, to a level that it is expected to become the leading global cause of death within a decade. While there are interventions available to reestablish coronary perfusion and treat arrhythmias associated with MI, there is no currently available approach to directly treat cardiomyocyte death. Developing novel therapeutic approaches that can compensate for the loss of cardiomyocytes and other cardiac resident cells will have broad translational potential in the treatment of MI to prevent or slow the progression into heart failure.

One such approach is the use of stem cells to regenerate cardiomyocytes lost due to MI. Despite the promise of these regenerative approaches there are several key challenges associated with the development of stem cells as a therapeutic approach for MI. First, only a small number of injected cells eventually implant into the infarct zone. It is common for therapeutic efforts to involve systemic or local injection of millions of stem cells into an animal to generate a very small number of viable cells that arrive and/or implant into the injured myocardium following MI. There are several different aspects that contribute to the loss of injected cells, varying from physical shear forces during the delivery and transit of stem cells through the circulatory system to the immunological effects that can result in the death of cells even before they arrive at the target tissues. Increasing the efficiency of delivery would reduce the number of stem cells that would have to be generated for each preparation, thus increasing the ability to translate experimental stem cell approaches into viable therapies. Second, exposure to systemic and local stressors results in the loss of a massive percentage of injected stem cells after implantation into the local tissue environment. These losses can result from the inflammatory effects that are greatly exacerbated in the infarct zone in the heart following MI. These losses of stem cells can decrease the efficacy of any attempted therapeutic approach, as there will be fewer cells that can actually contribute to the regeneration of the heart. Third, cells that do implant have to differentiate into cell types that have some restorative effect on heart structure and function. There is significant disagreement on the extent that many of these various stem cell types can differentiate into functional cardiomyocytes. Pluripotent stem cells are believed to have the best capability of differentiating into cardiac cells within an appropriate developmental niche. Fourth, the use of (particularly pluripotent) stem cells can raise concerns about potential tumorigenesis following injection of the cells. While the degree of risk for tumor formation can vary by type of stem cells and the specific approach used, the issue of tumor formation can complicate the use of any stem cells in regenerative medicine.

Results

Because the formation of teratomas containing tissue from all three germ layers has been a major concern of using ES cells for regenerative medicine, the ES cell aggregates were pre-differentiated in the prehatching embryo-like microcapsules for 3 days using BMP-4 (25 ng/ml) and bFGF (5 ng/ml) to direct them into the mesodermal early cardiac lineage before transplantation into animals. Successful pre-differentiation was confirmed by qRT-PCR data showing that expression of the mesoderm (GSC) and cardiac (Nkx2.5) marker genes were significantly higher in the pre-differentiated cells compared to the aggregated ES cells before differentiation. The expression of cardiac muscle gene marker (cTnT) was also up regulated while the pluripotency marker gene (Oct-4, significantly different) together with the ectoderm (nestin) and endoderm (Sox-7) marker genes were down regulated in the pre-differentiated cells.

After pre-differentiation, the aggregates were released from the core-shell microcapsules using isotonic sodium citrate solution to mimic the physiologic process of the release of blastocyst from the zona pellucida (embryo shell) for further differentiation. Next the released aggregates were encapsulated in a biodegradable and biocompatible alginate-chitosan micro-matrix (ACM) by soaking the aggregates first in chitosan (0.4% w/v) and then in oxidized alginate (0.15% w/v) solution and the soaking procedure was repeated an additional time. The idea here is that chitosan is a positively charged polymer that can be attracted to the cells in the aggregate because the plasma membrane of mammalian cells is negatively charged. The same electrostatic interaction applies to the coating of alginate on chitosan and vice versa because alginate is a negatively charged natural polymer. This biodegradable ACM should maintain the integrity of each aggregate during injection through a syringe and help retain the cells at the site of injection after implantation.

Successful ACM encapsulation of the pre-differentiated aggregates using the aforementioned procedure was first evidenced by phase and confocal fluorescence micrographs showing a matrix with green fluorescence throughout the aggregates as a result of labeling the alginate for encapsulation with a green fluorescence probe (FITC). It was further confirmed by the difference in morphology of ACM encapsulated versus non-encapsulated cell aggregates cultured in Petri dish at different days: all the cell aggregates were integral on day 0; cells detached from the non-encapsulated aggregates, spread out, and attached on the surface of petri dish on day 1, which was not the case for the ACM encapsulated aggregates; and cells detached from the ACM encapsulated aggregates as a result of degradation of the oxidized alginate in ACM and attached on the petri dish surface on day 3, similarly to that of non-encapsulated cells on day 1. A major advantage of ACM encapsulation is that it does not significantly increase the aggregate size.

### Example 5

### ACM Encapsulated Pre-Differentiated ES Cell Aggregates Minimizes Fibrosis and Tumor Formation in Mouse Myocardial Infarction (MI) Model Created by Permanent Ligation (PLA)

For initial proof of concept studies with ACM encapsulated cell aggregates a permanent ligation mouse model of MI was used with intramyocardial injection of with 2×10⁵ cells/animal into the periphery of the infarct zone immediately following permanent occlusion of the LAD.

Being consistent with the literature, injection of non-encapsulated cells (even with pre-differentiation to the cardiac lineage) resulted in obvious tumor formation in 7 out of 8 animals. Interestingly, microscopic examination of the tumor architecture revealed that it is a diffuse tissue with loose extracellular matrix rather than typical teratoma with dense tissue architecture, which might be a result of pre-differentiation of the ES cell aggregates into a mesoderm lineage before injection. More importantly, tumor formation was not observed in any of the 8 animals treated with the ACM encapsulated cell aggregates. In other words, ACM encapsulation of the pre-differentiated cell aggregates could effectively depress tumor formation, potentially addressing one of the major concerns with using pluripotent stem cells for regenerative medicine. Moreover, cardiomyocyte-like cells possibly differentiated from the transplanted cells were observable in the MI tissue treated with the ACM encapsulated cell aggregates.

Besides the absence of tumor formation, the MI tissue treated with encapsulated cell aggregates also had reduced total area of fibrosis while it was extensive in both the saline and materials (ACM without cells) treated groups. These differences between the groups treated with and without the pre-differentiated cell aggregates were further quantified to be statistically significant.

### Example 6

### ACM Encapsulated Pre-Differentiated ES Cell Aggregates Improves Cardiac Function and Animal Survival in Mouse Myocardial Infarction (MI) Model Created by Permanent Ligation (PLA)

Additional studies tested cardiac function that reflects the contractility of the left ventricular wall by measuring the left ventricle inner diameter during both systole (LVIDs) and diastole (LVIDd) using echocardiography. The treatment with encapsulated cell aggregates significantly reduced both LIVDs and LVIDd compared to the treatments with saline and ACM in the mouse MI model although they were still larger than that of normal heart with no PLA.

Treatment with non-encapsulated ES cell aggregates significantly reduced both LIVDs and LVIDd compared to the treatments with saline and ACM in the mouse MI model. However, the high rate (⅞ or 87.5%) of forming giant tumors led to the worst survival percentage and survival time among the four treatment groups with PLA. The mice with MI treated with nonencapsulated started to die on day 2 while it did not happen to the ACM and saline treated mice until day 5 and the ACM encapsulated cell aggregates treated mice until day 8. Therefore, some of the ‘protective’ function for the non-encapsulated stem cells likely comes from the mouse dying from the effects of the large tumor before additional cardiac structural damage could develop. On day 15, the percentage survival of the MI mice treated with ACM encapsulated cell aggregates was more than twice of that for the other three treatments. Moreover, the survival time of animals following PLA was significantly longer for the mice treated with ACM encapsulated cell aggregates compared to non-encapsulated cell aggregates.

### Example 7

### ACM Encapsulated Cells Differentiated into Cardiac-Like Cells after Intramyocardial Injection

To explore if the significantly improved survival time of the MI mice treated with the ACM encapsulated cell aggregates is a result of their regenerative capability of further differentiating into cardiac-like cells after injection, the cells were labeled in the encapsulated aggregates with DiI and sacrificed the mice on day 15 for analysis to see if the DiI-labeled cells also express cardiac cell markers including cTnT in the heavy chain of cardiac muscle, α-actinin in the z line between cardiac sarcomeres, and connexin 43 in the gap junctions between cardiomyocytes. Extensive co-localization of DiI and all the three cardiac cell markers was evident. The data suggests the regenerative capability of the encapsulated cell aggregates and may be responsible for the improved survival rate and significantly extended survival time of the MI mice treated with ACM encapsulated cell aggregates.

### Example 8

### Microencapsulation to Enrich Cancer Stem-Like Cells (CSCs)

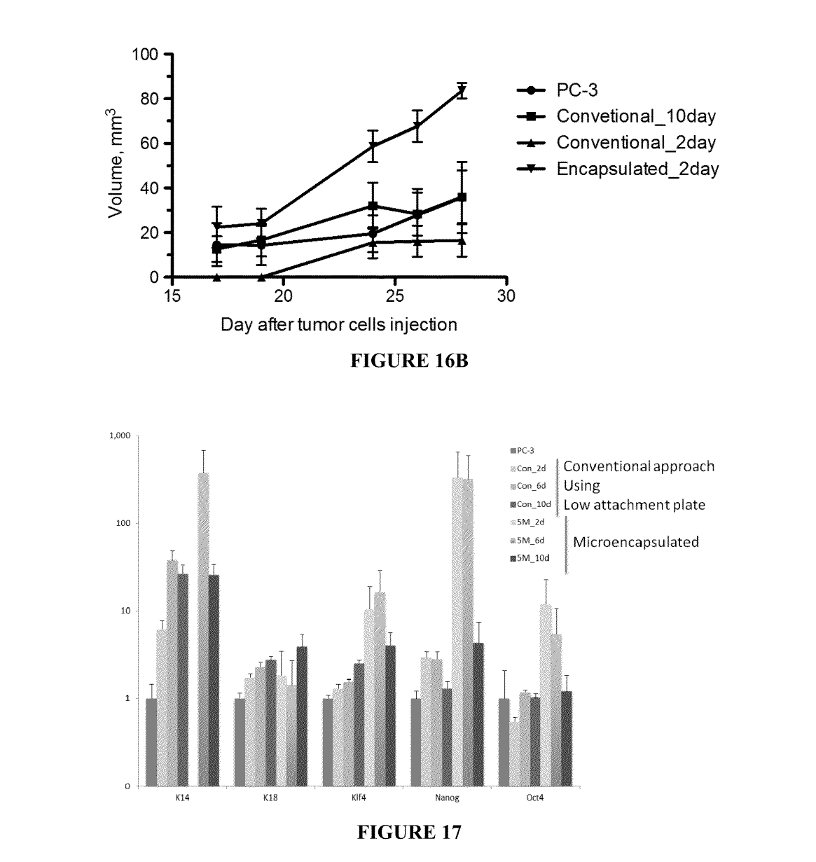

FIGS. 16A-16B show that cancer stem cells (CSCs) enriched by microencapsulation using the aqueous liquid core and alginate hydrogel shell are much more tumorigenic, compared to that obtained using the conventional approach using expensive ultralow attachment plate. FIGS. 16A and 16B show tumor incidence (%) (FIG. 16A) and tumor volume (mm³) (FIG. 16B) in mice injected with 3,000 cells from the following conditions: PC-3 control CSCs, CSCs after convention culture for 10 days or 2 days, and encapsulated CSCs cultured for 2 days.

FIG. 17 is qRT-PCR data of pluripotency genes showing that the aggregated CSCs cells in 3D liquid core have significantly higher expression of pluripotency genes compared to PC-3 cells or CSCs cultured using conventional methods.

Moreover, with the microencapsulation approach, the CSCs can be enriched in only two days while the conventional approach requires usually 10 days, greatly speed up the process. Therefore, the microencapsulation approach for enriching CSCs could be tremendous for identifying effective therapies both in research lab and in clinical settings to eliminate cancer from its root-the CSCs.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. Microcapsules comprising
(a) a core comprising a mammalian cell or cell aggregate suspended or encapsulated in a matrix; and
(b) a shell surrounding the core comprising a biocompatible hydrogel,
wherein the matrix and biocompatible hydrogel are distinct in their chemical composition.

2. The microcapsules of claim 1, wherein the matrix comprises a viscous aqueous liquid.

3. The microcapsules of claim 1, wherein the matrix comprises a hydrogel.

4. The microcapsules of claim 1, wherein the shell comprises alginate or a derivative thereof.

5. The microcapsules of claim 1, wherein the mammalian cell or cell aggregate is a pluripotent stem cell, multipotent stem cell, progenitor cell, primary cell, or gamete.

6. The microcapsules of claim 1, wherein the mammalian cell or cell aggregate comprises endothelial cells, pericytes, mesenchymal stem cells, or a combination thereof.

7. The microcapsules of claim 1, wherein the mammalian cell or cell aggregate comprises a pluripotent stem cell and the matrix comprises a viscous aqueous liquid.

8. The microcapsules of claim 1, wherein the microcapsules have an average diameter ranging from about 50 microns to about 1000 microns.

9. The microcapsules of claim 1, wherein the shell has a thickness ranging from about 10 microns to about 100 microns.

10. The microcapsules of claim 1, wherein the core, shell, or combinations thereof further comprise a bioactive agent.

11. The microcapsules of claim 10, wherein the bioactive agent comprises a biomolecule.

12. A microfluidic device comprising
(a) a core inlet channel, a first shell inlet channel, a second shell inlet channel, a first crosslinker inlet channel, and a second crosslinker inlet channel, all of which fluidly converge to form a flow focusing junction; and
(b) an outlet channel flowing from the flow focusing junction;
wherein the core inlet channel is positioned between the first shell inlet channel and the second shell inlet channel;
wherein the core inlet channel, the first shell inlet channel, and the second shell inlet channel are positioned between the first crosslinker inlet channel and the second crosslinker inlet channel;
wherein the distance between the core inlet channel and the first shell inlet channel is substantially equal to the distance between the core inlet channel and the second shell inlet channel;
wherein the distance between the core inlet channel and the first crosslinker inlet channel is substantially equal to the distance between the core inlet channel and the second crosslinker inlet channel;
wherein the distance between the core inlet channel and the first crosslinker inlet channel and the distance between the core inlet channel and the second crosslinker inlet channel is less than about 15 times the width of the core inlet channel.

13. The device of claim 12, wherein the core inlet channel has a width of between about 50 microns and about 300 microns.

14. The device of claim 12, wherein the core inlet channel has a height of between about 50 microns and about 300 microns.

15. The device of claim 12, wherein the width of the first shell inlet channel and the width of the second shell inlet channel are between about 15% and about 95% of the width of the core inlet channel.

16. The device of claim 12, wherein the height of the first shell inlet channel and the height of the second shell inlet channel are between about 100% and about 300% of the height of the core inlet channel.

17. The device of claim 12, wherein the width of the first crosslinker inlet channel and the width of the second crosslinker inlet channel are between about 15% and about 600% of the width of the core inlet channel.

18. The device of claim 12, wherein the height of the first crosslinker inlet channel and the height of the second crosslinker inlet channel are between about 15% and about 500% of the height of the core inlet channel.

19. A method of forming microcapsules, comprising
(a) flowing an aqueous solution comprising cells or cell aggregates suspended in a matrix through the core inlet channel of the device of claim 12;
(b) flowing an aqueous solution comprising biocompatible hydrogel-forming material through the first shell inlet channel and the second shell inlet channel of the device of claim 12; and
(c) flowing an organic solution, dispersion, or emulsion comprising a crosslinking agent through the first crosslinker inlet channel and the second crosslinker inlet channel of the device of claim 12.

20. A method of encapsulating cells comprising
(a) providing an electrospray apparatus comprising
i. a coaxial needle comprising a central lumen surrounded by an outer lumen;
ii. a gelling bath comprising a crosslinking agent; and
iii. a power source configure to provide a potential bias between the coaxial needle and the gelling bath;
wherein the coaxial needle is configured such that fluids ejected from the coaxial needle contact the gelling bath; and
(b) simultaneously ejecting an aqueous solution comprising cells or cell aggregates suspended in a matrix from the central lumen and an aqueous solution comprising biocompatible hydrogel-forming material from the outer lumen.
